(19) United States
(12) Patent Application Publication
(10) Pub. No.: US 2024/0042410 A1
(43) Pub. Date: Feb. 8, 2024

(54) PROCESS FOR PREPARING MICROCAPSULES

(71) Applicant: FIRMENICH SA, Satigny (CH)

(72) Inventors: Huda JERRI, Plainsboro, NJ (US); Christopher HANSEN, Plainsboro, NJ (US); Nicholas IMPELLIZZERI, Plainsboro, NJ (US); Amal ELABBADI, Satigny (CH); Marlene JACQUEMOND, Satigny (CH); Philipp ERNI, Satigny (CH)

(21) Appl. No.: 18/462,228

(22) Filed: Sep. 6, 2023

Related U.S. Application Data
(62) Division of application No. 16/975,042, filed on Aug. 21, 2020.
(60) Provisional application No. 62/687,876, filed on Jun. 21, 2018.

(30) Foreign Application Priority Data
Jun. 21, 2018 (EP) 18179125.2
Jul. 18, 2018 (EP) 18184284.0

(51) Int. Cl. B01J 13/18; A23L 27/00; A23P 10/35; A61K 8/11; A61K 8/19; A61K 8/87; A61K 8/98; A61Q 5/02; A61Q 5/12; A61Q 11/00; A61Q 15/00; A61Q 19/10; B01J 13/14; B01J 13/22; C11B 9/00; C11D 3/00; C11D 3/06; C11D 3/10; C11D 3/37; C11D 3/384; C11D 3/50; C11D 11/00

(52) U.S. Cl. CPC B01J 13/18; A23L 27/72; A23P 10/35; A61K 8/11; A61K 8/19; A61K 8/87; A61K 8/986; A61Q 5/02; A61Q 5/12; A61Q 11/00; A61Q 15/00; A61Q 19/10; B01J 13/14; B01J 13/22; C11B 9/0003; C11D 3/0015; C11D 3/06; C11D 3/10; C11D 3/3726; C11D 3/384; C11D 3/505; C11D 11/0017; C11D 11/0023; A23V 2002/00; A61K 2800/621; A61K 2800/654

(57) ABSTRACT

The present invention relates to a new process for the preparation of core-shell microcapsules. Microcapsules are also an object of the invention. Consumer products comprising said microcapsules, in particular perfumed consumer products or flavoured consumer products are also part of the invention.

### CROSS-REFERENCE TO RELATED APPLICATIONS

This application is a divisional of U.S. application Ser. No. 16/975,042, which is the U.S. National Phase Application of International Patent Application No. PCT/EP2019/066215, filed Jun. 19, 2019, which claims the benefit of priority to U.S. Provisional Patent Application No. 62/687,876, filed Jun. 21, 2018, to European Patent Application No. 18179125.2, filed Jun. 21, 2018, and to European Patent Application No. 18184284.0, filed Jul. 18, 2018, the entire contents of each of which are hereby incorporated by reference herein.

### TECHNICAL FIELD

The present invention relates to a new process for the preparation of core-shell microcapsules. Microcapsules are also an object of the invention. Consumer products comprising said microcapsules, in particular perfumed consumer products or flavoured consumer products are also part of the invention.

### BACKGROUND OF THE INVENTION

One of the problems faced by the perfume and flavour industry lies in the relatively rapid loss of olfactive benefit provided by active compounds due to their volatility. The encapsulation of those active substances provides at the same time a protection of the ingredients there-encapsulated against “aggressions” such as oxidation or moisture and allows, on the other hand, a certain control of the kinetics of flavour or fragrance release to induce sensory effects through sequential release.

Polyurea and polyurethane-based microcapsule slurry are widely used for example in perfumery industry for instance as they provide a long lasting pleasant olfactory effect after their applications on different substrates. Those microcapsules have been widely disclosed in the prior art (see for example WO2007/004166 or EP 2300146 from the Applicant).

Therefore, there is still a need to provide new microcapsules while not compromising on their performance, in particular in terms of stability in a consumer product, as well as in delivering a good performance in terms of hydrophobic material delivery.

The present invention is proposing a solution to the above-mentioned problem, based on new core-shell microcapsules comprising a cross-linked biopolymer shell.

### SUMMARY OF THE INVENTION

It has now been found that performing microcapsules encapsulating hydrophobic materials, preferably active ingredients could be obtained by salt complexation of proteins to densify the membrane, followed by a crosslinking of the protein. The process of the invention therefore provides a solution to the above-mentioned problems as it allows preparing microcapsules with the desired stability in different applications.

In a first aspect, the present invention relates to a process for preparing a core-shell microcapsule slurry, wherein the process comprises the steps of:
(i) Admixing a salt and optionally a cross-linker into an aqueous solution comprising at least one protein to form an aqueous phase;
(ii) Dispersing an oil phase comprising an hydrophobic material, preferably a perfume oil or a flavor oil, into the aqueous phase to form an oil-in-water emulsion;
(iii) Adding into the oil-in-water emulsion a cross-linker if such a cross-linker has not yet been added in step (i);
(iv) Applying sufficient conditions to induce the cross-linking of the protein so as to form a core-shell microcapsule in the form of a slurry.

In a second aspect, the invention relates to a core-shell microcapsules slurry comprising at least one microcapsules made of:
an oil-based core
optionally an inner shell made of a polymerized polyfunctional monomer;
a biopolymer shell comprising a protein, wherein at least one protein is cross-linked; and
optionally at least an outer mineral layer.

In a third aspect, the invention relates to a core-shell microcapsule slurry obtainable by the process as defined above.

In a fourth and fifth aspects, the invention relates to perfumed consumer products and flavoured edible products comprising the microcapsules defined above.

### BRIEF DESCRIPTION OF THE FIGURES

FIG. 1 represents the stability of the microcapsules of the invention in a shower gel base (37° C.-1 week).

FIG. 2 represents the stability of the microcapsules of the invention in a fabric softener base (37° C.-1 week).

FIG. 3 represents the olfactive performance of the microcapsules of the invention evaluated on blotters from a fabric softener base (37° C.-2 weeks).

FIG. 4 represents the stability of the microcapsules of the invention in a fabric softener base (37° C.-1 month).

FIG. 5 represents the olfactive performance of the microcapsules of the invention evaluated on line dried towels from a fabric softener base.

FIG. 6 represents scanning electron micrographs of mineralized microcapsules according to the invention, mineralized capsule K

FIG. 7 represents scanning electron micrographs of mineralized microcapsules according to the invention, mineralized capsule N

FIG. 8 represents scanning electron micrographs of mineralized microcapsules according to the invention, mineralized capsule O

FIG. 9 represents scanning electron micrographs of microcapsules according to the invention, capsule E

FIG. 10 represents scanning electron micrographs of mineralized microcapsules P subjected to spray drying protocol, according to the invention mineralized and spray dried capsules P

FIG. 11 represents scanning electron micrographs of capsules J according to the invention.

FIG. 12 represents olfactive performance of the invention's microcapsules in an antiperspirant roll-on composition evaluated on blotters.

FIG. 13 represents olfactive performance of the invention's microcapsules in leave-on conditioner composition evaluated on hair.

FIG. 14 represents olfactive performance of the invention's microcapsules in rinse-off shampoo composition evaluated on hair.

FIG. 15 represents the percentage of microcapsule deposition of microcapsules according to the invention (Capsules E, G, H) as well as mineralized microcapsules according to the invention (Capsules N, K, L) onto hair from a model surfactant mixture.

FIG. 16 represents the stability of the mineral coating on microcapsules according to the invention (Capsule N) in hydrogen peroxide solutions (pH 6.5) after 1 month incubation at 22° C.

FIG. 17 represents an average olfactive intensities of high ethanol EdT compositions demonstrating before and after rubbing effects.

FIG. 18 represents an average olfactive intensities of low ethanol EdT compositions demonstrating before and after rubbing effects.

### DETAILED DESCRIPTION OF THE INVENTION

Unless stated otherwise, percentages (%) are meant to designate a percentage by weight of a composition.

By “Hydrophobic material”, it is meant a material which forms a two-phase dispersion when mixed with water. According to the invention, the hydrophobic material can be “inert” material like solvents or active ingredients. According to an embodiment, the hydrophobic material is a hydrophobic active ingredient.

By “active ingredient”, it is meant a single compound or a combination of ingredients.

By “perfume oil or flavour oil”, it is meant a single perfuming or flavouring compound or a mixture of several perfuming or flavouring compounds.

By “consumer product” or “end-product” it is meant a manufactured product ready to be distributed, sold and used by a consumer.

For the sake of clarity, by the expression “dispersion” in the present invention it is meant a system in which particles are dispersed in a continuous phase of a different composition and it specifically includes a suspension or an emulsion.

A “core-shell microcapsule”, or the similar, in the present invention it is meant that capsules have a particle size distribution in the micron range (e.g. a mean diameter (d(v, 0.5)) preferably comprised between about 1 and 3000 microns) and comprise a biopolymer shell and an internal continuous oil phase enclosed by the biopolymer shell. According to the invention, the wordings “mean diameter” or “mean size” are used indifferently.

Microcapsules of the present invention have a mean size preferably greater than 10 microns, more preferably greater than 15 microns, even more preferably greater than 20 microns.

According to an embodiment, microcapsules have a mean size comprised between 10 and 500 microns, preferably between 10 and 100 microns, more preferably between 10 and 50 microns.

According to an embodiment, microcapsules have a mean size comprised between 15 and 500 microns, preferably between 15 and 100 microns, more preferably between 15 and 50 microns.

According to an embodiment, microcapsules have a mean size comprised between 20 and 500 microns, preferably between 20 and 100 microns, more preferably between 20 and 50 microns.

Microcapsules according to the invention are preferably not agglomerated.

By “biopolymer membrane” or “biopolymer shell”, it is meant a layer comprising crosslinked proteins, preferably enzymatically crosslinked.

In the context of the invention, a “mineral layer” is composed of a stable inorganic mineral phase that grows normal to the terminating charged surface of the shell to yield a textured mineral surface.

According to an embodiment, capsules according to the present invention are organic-inorganic hybrid capsules. According to this particular embodiment, an orthosilicate, a silane or a combination of silanes can be added from the oil phase or the water phase to form a hybridized inorganic/organic membrane or surface coating. Silanes can be suspended in the oil phase to silicify the inner membrane, or can be added post-emulsification to form a silicified shell around the burgeoning polymeric capsule membrane. Inside-out and outside-in sol gel polymerization can occur by forming and hardening 3D siloxane bonds inside or outside the polymer membrane via condensation of alkoxide in or on the emulsion droplets.

By “mineral precursor”, it should be understood a mineral precursor required for growth of the desired phase. The mineral precursor is preferably a mineral water-soluble salt containing at least one part of the necessary ions for growth of the desired mineral phase.

The terminology of “incubating” is used in the context of the present invention to describe the act of submerging the microcapsules in the precursor solution and allowing it time to interact with the microcapsules.

By “polyfunctional polymer”, it is meant a molecule that, as a unit, reacts or binds chemically to form a polymer or supramolecular polymer. The polyfunctional polymer of the invention has at least two functions capable of forming a microcapsule shell.

By “polyurea-based” inner wall or inner shell, it is meant that the polymer comprises urea linkages produced by either an amino-functional crosslinker or hydrolysis of isocyanate groups to produce amino groups capable of further reacting with isocyanate groups during interfacial polymerization.

By “polyurethane-based” inner wall or inner shell, it is meant that the polymer comprises urethane linkages produced by reaction of a polyol with the isocyanate groups during interfacial polymerization.

By “protein”, it is meant a single protein or a combination of proteins.

### Process for Preparing a Core-Shell Microcapsule Slurry

The present invention therefore relates in a first aspect to a process for preparing a core-shell microcapsule slurry, wherein the process comprises the steps of:
(i) Admixing a salt and optionally a cross-linker into an aqueous solution comprising a protein to form an aqueous phase;
(ii) Dispersing an oil phase comprising a hydrophobic material, preferably a perfume oil or a flavor oil, into the aqueous phase to form an oil-in-water emulsion;
(iii) Adding into the oil-in-water emulsion a cross-linker if such a cross-linker has not yet been added in step (i);
(iv) Applying sufficient conditions to induce the cross-linking of the protein so as to form a core-shell microcapsule in the form of a slurry.
   According to an embodiment, step (iv) consists of applying sufficient conditions to induce the cross-linking of the protein by the cross-linker so as to form a core-shell microcapsule in the form of a slurry.

### Hydrophobic Material (Oil Phase)

According to an embodiment, the hydrophobic material is a hydrophobic active ingredient. According to a preferred embodiment, the active ingredient comprises a perfume oil or a flavour oil. Alternative ingredients which could benefit from being encapsulated could be used either instead of a perfume or flavour, or in combination with a perfume or flavour. Non-limiting examples of such ingredients include a cosmetic, skin caring, malodour counteracting, bactericide, fungicide, pharmaceutical or agrochemical ingredient, a sanitizing agent, an insect repellent or attractant, and mixture thereof.

The nature and type of the insect repellent or attractant that can be present in the hydrophobic internal phase do not warrant a more detailed description here, which in any case would not be exhaustive, the skilled person being able to select them on the basis of its general knowledge and according to the intended use or application.

Examples of such insect repellent or attractant are birch, DEET (N,N-diethyl-m-toluamide), essential oil of the lemon eucalyptus (Corymbia citriodora) and its active compound p-menthane-3,8-diol(PMD), icaridin (hydroxyethyl isobutyl piperidine carboxylate), Nepelactone, Citronella oil, Neem oil, Bog Myrtle (Myrica Gale), Dimethyl carbate, Tricyclodecenyl allyl ether, IR3535 (3-[N-Butyl-N-acetyl]-aminopropionic acid, ethyl ester, Ethylhexanediol, Dimethyl phthalate, Metofluthrin, Indalone, SS220, anthranilate-based insect repellents, and mixtures thereof.

By “perfume oil” (or also “perfume”) or “flavour” what is meant here is an ingredient or composition that is a liquid at about 20° C. Said perfume or flavour oil can be a perfuming or flavouring ingredient alone or a mixture of ingredients in the form of a perfuming or flavouring composition. As a “perfuming ingredient” it is meant here a compound, which is used in perfuming preparations or compositions to impart as primary purpose a hedonic effect. In other words such an ingredient, to be considered as being a perfuming one, must be recognized by a person skilled in the art as being able to at least impart or modify in a positive or pleasant way the odor of a composition, and not just as having an odor. The nature and type of the perfuming ingredients present in the oil phase do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said ingredients may also be compounds known to release in a controlled manner various types of perfuming compounds.

The perfuming ingredients may be dissolved in a solvent of current use in the perfume industry. The solvent is preferably not an alcohol. Examples of such solvents are diethyl phthalate, isopropyl myristate, Abalyn® (rosin resins, available from Eastman), benzyl benzoate, ethyl citrate, limonene or other terpenes, or isoparaffins. Preferably, the solvent is very hydrophobic and highly sterically hindered, like for example Abalyn® or benzyl benzoate. Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

Preferred perfuming ingredients are those having a high steric hindrance and in particular those from one of the following groups:
Group 1: perfuming ingredients comprising a cyclohexane, cyclohexene, cyclohexanone or cyclohexenone ring substituted with at least one linear or branched C₁ to C₄ alkyl or alkenyl substituent;
Group 2: perfuming ingredients comprising a cyclopentane, cyclopentene, cyclopentanone or cyclopentenone ring substituted with at least one linear or branched C₄ to C₈ alkyl or alkenyl substituent;
Group 3: perfuming ingredients comprising a phenyl ring or perfuming ingredients comprising a cyclohexane, cyclohexene, cyclohexanone or cyclohexenone ring substituted with at least one linear or branched C₅ to C₈ alkyl or alkenyl substituent or with at least one phenyl substituent and optionally one or more linear or branched C₁ to C₃ alkyl or alkenyl substituents;
Group 4: perfuming ingredients comprising at least two fused or linked C₅ and/or C₆ rings;
Group 5: perfuming ingredients comprising a camphor-like ring structure;
Group 6: perfuming ingredients comprising at least one C7 to C20 ring structure;
Group 7: perfuming ingredients having a logP value above 3.5 and comprising at least one tert-butyl or at least one trichloromethyl substitutent;

Examples of ingredients from each of these groups are:
Group 1: 2,4-dimethyl-3-cyclohexene-1-carbaldehyde (origin: Firmenich SA, Geneva, Switzerland), isocyclocitral, menthone, isomenthone, Romascone ° (methyl 2,2-dimethyl-6-methylene-1-cyclohexanecarboxylate, origin: Firmenich SA, Geneva, Switzerland), nerone, terpineol, dihydroterpineol, terpenyl acetate, dihydroterpenyl acetate, dipentene, eucalyptol, hexylate, rose oxide, Perycorolle® ((S)-1,8-p-menthadiene-7-ol, origin: Firmenich SA, Geneva, Switzerland), 1-p-menthene-4-ol, (1RS,3RS,4SR)-3-p-mentanyl acetate, (1R,2S,4R)-4,6,6-trimethyl-bicyclo[3,1,1]heptan-2-ol, Doremox® (tetrahydro-4-methyl-2-phenyl-2H-pyran, origin: Firmenich SA, Geneva, Switzerland), cyclohexyl acetate, cyclanol acetate, Fructalate® (1,4-cyclohexane diethyldicarboxylate, origin: Firmenich SA, Geneva, Switzerland), Koumalactone® ((3ARS,6SR,7ASR)-perhydro-3,6-dimethyl-benzo[B]furan-2-one, origin: Firmenich SA, Geneva, Switzerland), Natactone® ((6R)-perhydro-3,6-dimethyl-benzo[B]furan-2-one, origin: Firmenich SA, Geneva, Switzerland), 2,4,6-trimethyl-4-phenyl-1,3-dioxane, 2,4,6-trimethyl-3-cyclohexene-1-carbaldehyde;
Group 2: (E)-3-methyl-5-(2,2,3-trimethyl-3-cyclopenten-1-yl)-4-penten-2-ol (origin: Givaudan SA, Vernier, Switzerland), (1′R,E)-2-ethyl-4-(2′,2′,3′-trimethyl-3′-cyclopenten-1′-yl)-2-buten-1-ol (origin: Firmenich SA, Geneva, Switzerland), Polysantol® ((1′R,E)-3,3-dimethyl-5-(2′,2′,3′-trimethyl-3′-cyclopenten-1′-yl)-4-penten-2-ol, origin: Firmenich SA, Geneva, Switzerland), fleuramone, Hedione® HC (methyl-cis-3-oxo-2-pentyl-1-cyclopentane acetate, origin: Firmenich SA, Geneva, Switzerland), Veloutone® (2,2,5-Trimethyl-5-pentyl-1-cyclopentanone, origin: Firmenich SA, Geneva, Switzerland), Nirvanol® (3,3-dimethyl-5-(2,2,3-trimethyl-3-cyclopenten-1-yl)-4-penten-2-ol, origin: Firmenich SA, Geneva, Switzerland), 3-methyl-5-(2,2,3-trimethyl-3-cyclopenten-1-yl)-2-pentanol (origin, Givaudan SA, Vernier, Switzerland);
Group 3: damascones, Neobutenone® (1-(5,5-dimethyl-1-cyclohexen-1-yl)-4-penten-1-one, origin: Firmenich SA, Geneva, Switzerland), nectalactone ((1′R)-2-[2-(4′-methyl-3′-cyclohexen-1′-yl)propyl]cyclopentanone), alpha-ionone, beta-ionone, damascenone, Dynascone® (mixture of 1-(5,5-dimethyl-1-cyclohexen-1-yl)-4-penten-1-one and 1-(3,3-dimethyl-1-cyclohexen-1-yl)-4-penten-1-one, origin: Firmenich SA, Geneva, Switzerland), Dorinone® beta (1-(2,6,6-trimethyl-1-cyclohexen-1-yl)-2-buten-1-one, origin: Firmenich SA, Geneva, Switzerland), Romandolide® ((1S,1′R)-[1-(3′,3′-Dimethyl-1′-cyclohexyl)ethoxycarbonyl]methyl propanoate, origin: Firmenich SA, Geneva, Switzerland), 2-tert-butyl-1-cyclohexyl acetate (origin: International Flavors and Fragrances, USA), Limbanol® (1-(2,2,3,6-tetramethyl-cyclohexyl)-3-hexanol, origin: Firmenich SA, Geneva, Switzerland), trans-1-(2,2,6-trimethyl-1-cyclohexyl)-3-hexanol (origin: Firmenich SA, Geneva, Switzerland), (E)-3-methyl-4-(2,6,6-trimethyl-2-cyclohexen-1-yl)-3-buten-2-one, terpenyl isobutyrate, Lorysia® (4-(1,1-dimethylethyl)-1-cyclohexyl acetate, origin: Firmenich SA, Geneva, Switzerland), 8-methoxy-1-p-menthene, Helvetolide® ((1S,1′R)-2-[1-(3′,3′-dimethyl-1′-cyclohexyl) ethoxy]-2-methylpropyl propanoate, origin: Firmenich SA, Geneva, Switzerland), para tert-butylcyclohexanone, menthenethiol, 1-methyl-4-(4-methyl-3-pentenyl)-3-cyclohexene-1-carbaldehyde, allyl cyclohexylpropionate, cyclohexyl salicylate, 2-methoxy-4-methylphenyl methyl carbonate, ethyl 2-methoxy-4-methylphenyl carbonate, 4-ethyl-2-methoxyphenyl methyl carbonate;
Group 4: Methyl cedryl ketone (origin: International Flavors and Fragrances, USA), Verdylate, vetyverol, vetyverone, 1-(octahydro-2,3,8,8-tetramethyl-2-naphtalenyl)-1-ethanone (origin: International Flavors and Fragrances, USA), (5RS,9RS,10SR)-2,6,9,10-tetramethyl-1-oxaspiro[4.5]deca-3,6-diene and the (5RS,9SR,10RS) isomer, 6-ethyl-2,10,10-trimethyl-1-oxaspiro[4.5]deca-3,6-diene, 1,2,3,5,6,7-hexahydro-1,1,2,3,3-pentamethyl-4-indenone (origin: International Flavors and Fragrances, USA), Hivernal® (a mixture of 3-(3,3-dimethyl-5-indanyl)propanal and 3-(1,1-dimethyl-5-indanyl)propanal, origin: Firmenich SA, Geneva, Switzerland), Rhubofix® (3′,4-dimethyl-tricyclo[6.2.1.0(2,7)]undec-4-ene-9-spiro-2′-oxirane, origin: Firmenich SA, Geneva, Switzerland), 9/10-ethyldiene-3-oxatricyclo[6.2.1.0(2,7)]undecane, Polywood® (perhydro-5,5,8A-trimethyl-2-naphthalenyl acetate, origin: Firmenich SA, Geneva, Switzerland), octalynol, Cetalox® (dodecahydro-3a,6,6,9a-tetramethyl-naphtho[2,1-b]furan, origin: Firmenich SA, Geneva, Switzerland), tricyclo[5.2.1.0(2,6)]dec-3-en-8-yl acetate and tricyclo[5.2.1.0(2,6)]dec-4-en-8-yl acetate as well as tricyclo[5.2.1.0(2,6)]dec-3-en-8-yl propanoate and tricyclo[5.2.1.0(2,6)]dec-4-en-8-yl propanoate, (+)-(1S,2S,3S)-2,6,6-trimethyl-bicyclo[3.1.1]heptane-3-spiro-2′-cyclohexen-4′-one;
Group 5: camphor, borneol, isobornyl acetate, 8-isopropyl-6-methyl-bicyclo[2.2.2]oct-5-ene-2-carbaldehyde, camphopinene, cedramber (8-methoxy-2,6,6,8-tetramethyl-tricyclo[5.3.1.0(1,5)]undecane, origin: Firmenich SA, Geneva, Switzerland), cedrene, cedrenol, cedrol, Florex® (mixture of 9-ethylidene-3-oxatricyclo[6.2.1.0(2,7)]undecan-4-one and 10-ethylidene-3-oxatricyclo[6.2.1.0(2,7)]undecan-4-one, origin: Firmenich SA, Geneva, Switzerland), 3-methoxy-7,7-dimethyl-10-methylene-bicyclo[4.3.1]decane (origin: Firmenich SA, Geneva, Switzerland);
Group 6: Cedroxyde® (trimethyl-13-oxabicyclo-[10.1.0]-trideca-4,8-diene, origin: Firmenich SA, Geneva, Switzerland), Ambrettolide LG ((E)-9-hexadecen-16-olide, origin: Firmenich SA, Geneva, Switzerland), Habanolide® (pentadecenolide, origin: Firmenich SA, Geneva, Switzerland), muscenone (3-methyl-(4/5)-cyclopentadecenone, origin: Firmenich SA, Geneva, Switzerland), muscone (origin: Firmenich SA, Geneva, Switzerland), Exaltolide® (pentadecanolide, origin: Firmenich SA, Geneva, Switzerland), Exaltone® (cyclopentadecanone, origin: Firmenich SA, Geneva, Switzerland), (1-ethoxyethoxy)cyclododecane (origin: Firmenich SA, Geneva, Switzerland), Astrotone, 4,8-cyclododecadien-1-one;
Group 7: Lilial® (origin: Givaudan SA, Vernier, Switzerland), rosinol.

Preferably, the perfume comprises at least 30%, preferably at least 50%, more preferably at least 60% of ingredients selected from Groups 1 to 7, as defined above. More preferably said perfume comprises at least 30%, preferably at least 50% of ingredients from Groups 3 to 7, as defined above. Most preferably said perfume comprises at least 30%, preferably at least 50% of ingredients from Groups 3, 4, 6 or 7, as defined above.

According to another preferred embodiment, the perfume comprises at least 30%, preferably at least 50%, more preferably at least 60% of ingredients having a logP above 3, preferably above 3.5 and even more preferably above 3.75.

Preferably, the perfume used in the invention contains less than 10% of its own weight of primary alcohols, less than 15% of its own weight of secondary alcohols and less than 20% of its own weight of tertiary alcohols. Advantageously, the perfume used in the invention does not contain any primary alcohols and contains less than 15% of secondary and tertiary alcohols.

According to an embodiment, the oil phase (or the oil-based core) comprises:
25-100 wt % of a perfume oil comprising at least 15 wt % of high impact perfume raw materials having a Log T<-4, and
0-75 wt % of a density balancing material having a density greater than 1.07 g/cm³.
   “High impact perfume raw materials” should be understood as perfume raw materials having a LogT<-4. The odor threshold concentration of a chemical compound is determined in part by its shape, polarity, partial charges and molecular mass. For convenience, the threshold concentration is presented as the common logarithm of the threshold concentration, i.e., Log [Threshold] (“LogT”).
   A “density balancing material” should be understood as a material having a density greater than 1.07 g/cm³ and having preferably low or no odor.
   The odor threshold concentration of a perfuming compound is determined by using a gas chromatograph (“GC”). Specifically, the gas chromatograph is calibrated to determine the exact volume of the perfume oil ingredient injected by the syringe, the precise split ratio, and the hydrocarbon response using a hydrocarbon standard of known concentration and chain-length distribution. The air flow rate is accurately measured and, assuming the duration of a human inhalation to last 12 seconds, the sampled volume is calculated. Since the precise concentration at the detector at any point in time is known, the mass per volume inhaled is known and hence the concentration of the perfuming compound. To determine the threshold concentration, solutions are delivered to the sniff port at the back-calculated concentration. A panelist sniffs the GC effluent and identifies the retention time when odor is noticed. The average across all panelists determines the odor threshold concentration of the perfuming compound. The determination of odor threshold is described in more detail in C. Vuilleumier et al., Multidimensional Visualization of Physical and Perceptual Data Leading to a Creative Approach in Fragrance Development, Perfume & Flavorist, Vol. 33, September, 2008, pages 54-61.
   The nature of high impact perfume raw materials having a Log T<-4 and density balancing material having a density greater than 1.07 g/cm³ are described in WO2018115250, the content of which are included by reference.

According to an embodiment, the high impact perfume raw materials having a Log T<-4 are selected from the list in Table A below.

TABLE A
high impact perfume raw materials having a Log T < −4
Perfume raw materials (Log T < −4)

(+−)-1-METHOXY-3-HEXANETHIOL
4-(4-HYDROXY-1-PHENYL)-2-BUTANONE
(+−)-2-(4-METHYL-3-CYCLOHEXEN-1-YL)-2-PROPANETHIOL
2-METHOXY-4-(1-PROPENYL)-1-PHENYL ACETATE
PYRAZOBUTYLE
3-PROPYLPHENOL
1-(3-METHYL-1-BENZOFURAN-2-YL)ETHANONE
2-(3-PHENYLPROPYL)PYRIDINE
1-(3,3-DIMETHYL-1-CYCLOHEXEN-1-YL)-4-PENTEN-1-ONE (A) +
1-(5,5-DIMETHYL-1-CYCLOHEXEN-1-YL)-4-PENTEN-1-ONE
(B)
1-(5,5-DIMETHYL-1-CYCLOHEXEN-1-YL)-4-PENTEN-1-ONE
(3RS,3ARS,6SR,7ASR)-PERHYDRO-3,6-DIMETHYL-
BENZO[B]FURAN-2-ONE (A) + (3SR,3ARS,6SR,7ASR)-
PERHYDRO-3,6-DIMETHYL-BENZO[B]FURAN-2-ONE (B)
(+−)-1-(5-ETHYL-5-METHYL-1-CYCLOHEXEN-1-YL)-4-PENTEN-
1-ONE
(1′S,3′R)-1-METHYL-2-[(1′,2′,2′-TRIMETHYLBICYCLO[3.1.0]HEX-
3′-YL)METHYL]CYCLOPROPYL}METHANOL
(+−)-3-MERCAPTOHEXYL ACETATE
(2E)-1-(2,6,6-TRIMETHYL-1,3-CYCLOHEXADIEN-1-YL)-2-
BUTEN-1-ONE
7-METHYL-2H-1,5-BENZODIOXEPIN-3(4H)-ONE
(2E,6Z)-2,6-NONADIEN-1-OL
(4Z)-4-DODECENAL
(+−)-4-HYDROXY-2,5-DIMETHYL-3(2H)-FURANONE
METHYL 2,4-DIHYDROXY-3,6-DIMETHYLBENZOATE
3-METHYLINDOLE
(+−)-PERHYDRO-4ALPHA,8ABETA-DIMETHYL-4A-
NAPHTHALENOL
PATCHOULOL
2-METHOXY-4-(1-PROPENYL)PHENOL
(+−)-5,6-DIHYDRO-4-METHYL-2-PHENYL-2H-PYRAN (A) +
TETRAHYDRO-4-METHYLENE-2-PHENYL-2H-PYRAN (B)
4-METHYLENE-2-PHENYLTETRAHYDRO-2H-PYRAN (A) + (+−)-
4-METHYL-2-PHENYL-3,6-DIHYDRO-2H-PYRAN (B)
4-HYDROXY-3-METHOXYBENZALDEHYDE
NONYLENIC ALDEHYDE
2-METHOXY-4-PROPYLPHENOL
(2Z)-3-METHYL-5-PHENYL-2-PENTENENITRILE (A) + (2E)-3-
METHYL-5-PHENYL-2-PENTENENITRILE (B)
1-(SPIRO[4.5]DEC-6-EN-7-YL)-4-PENTEN-1-ONE (A) + 1-
(SPIRO[4.5]DEC-7-EN-7-YL)-4-PENTEN-1-ONE (B)
2-METHOXYNAPHTHALENE
(−)-(3AR,5AS,9AS,9BR)-3A,6,6,9A-
TETRAMETHYLDODECAHYDRONAPHTHO[2,1-B]FURAN
5-NONANOLIDE
(3AR,5AS,9AS,9BR)-3A,6,6,9A-
TETRAMETHYLDODECAHYDRONAPHTHO[2,1-B]FURAN
7-ISOPROPYL-2H,4H-1,5-BENZODIOXEPIN-3-ONE
COUMARIN
4-METHYLPHENYL ISOBUTYRATE
(2E)-1-(2,6,6-TRIMETHYL-1,3-CYCLOHEXADIEN-1-YL)-2-
BUTEN-1-ONE
BETA,2,2,3-TETRAMETHYL-DELTA-METHYLENE-3-
CYCLOPENTENE-1-BUTANOL
DELTA DAMASCONE ((2E)-1-[(1RS,2SR)-2,6,6-TRIMETHYL-3-
CYCLOHEXEN-1-YL]-2-BUTEN-1-ONE)
(+−)-3,6-DIHYDRO-4,6-DIMETHYL-2-PHENYL-2H-PYRAN
ANISALDEHYDE
PARACRESOL
3-ETHOXY-4-HYDROXYBENZALDEHYDE
METHYL 2-AMINOBENZOATE
ETHYL METHYLPHENYLGLYCIDATE
OCTALACTONE G
ETHYL 3-PHENYL-2-PROPENOATE
(−)-(2E)-2-ETHYL-4-[(1R)-2,2,3-TRIMETHYL-3-CYCLOPENTEN-1-
YL]-2-BUTEN-1-OL
PARACRESYL ACETATE
DODECALACTONE
TRICYCLONE
(+)-(3R,5Z)-3-METHYL-5-CYCLOPENTADECEN-1-ONE
UNDECALACTONE
(1R,4R)-8-MERCAPTO-3-P-MENTHANONE
(3S,3AS,6R,7AR)-3,6-DIMETHYLHEXAHYDRO-1-BENZOFURAN-
2(3H)-ONE
BÉTA IONONE
(+−)-6-PENTYLTETRAHYDRO-2H-PYRAN-2-ONE
(3E,5Z)-1,3,5-UNDECATRIENE
10-UNDECENAL (A) + (9E)-9-UNDECENAL (B) + (9Z)-9-
UNDECENAL (C)
(Z)-4-DECENAL
(+−)-ETHYL 2-METHYLPENTANOATE
1,2-DIALLYLDISULFANE
(2Z)-2-TRIDECENENITRILE (A) + (3Z)-3-
TRIDECENENITRILE (B) + (3E)-3-TRIDECENENITRILE (C) +
(2E)-2-TRIDECENENITRILE (D)
(+−)-2-ETHYL-4,4-DIMETHYL-1,3-OXATHIANE
(+)-(3R,5Z)-3-METHYL-5-CYCLOPENTADECEN-1-ONE
3-(4-TERT-BUTYLPHENYL)PROPANAL
ALLYL (CYCLOHEXYLOXY)ACETATE
METHYLNAPHTHYLKETONE
(+−)-(4E)-3-METHYL-4-CYCLOPENTADECEN-1-ONE (A) + (+−)-
(5E)-3-METHYL-5-CYCLOPENTADECEN-1-ONE (B) + (+−)-(5Z)-3-
METHYL-5-CYCLOPENTADECEN-1-ONE (C)
CYCLOPROPYLMETHYL (3Z)-3-HEXENOATE (A) +
CYCLOPROPYLMETHYL (3E)-3-HEXENOATE (B)
(4E)-4-METHYL-5-(4-METHYLPHENYL)-4-PENTENAL
(+−)-1-(5-PROPYL-1,3-BENZODIOXOL-2-YL)ETHANONE
4-METHYL-2-PENTYLPYRIDINE
(+−)-(E)-3-METHYL-4-(2,6,6-TRIMETHYL-2-CYCLOHEXEN-1-
YL)-3-BUTEN-2-ONE
(3ARS,5ASR,9ASR,9BRS)-3A,6,6,9A-
TETRAMETHYLDODECAHYDRONAPHTHO[2,1-B]FURAN
(2S,5R)-5-METHYL-2-(2-PROPANYL)CYCLOHEXANONE OXIME
6-HEXYLTETRAHYDRO-2H-PYRAN-2-ONE
(+−)-3-(3-ISOPROPYL-1-PHENYL)BUTANAL
METHYL 2-((1RS,2RS)-3-OXO-2-
PENTYLCYCLOPENTYL)ACETATE (A) + METHYL 2-((1RS,2SR)-
3-OXO-2-PENTYLCYCLOPENTYL)ACETATE (B)
1-(2,6,6-TRIMETHYL-1-CYCLOHEX-2-ENYL)PENT-1-EN-3-ONE
INDOL
7-PROPYL-2H,4H-1,5-BENZODIOXEPIN-3-ONE
ETHYL PRALINE
(4-METHYLPHENOXY)ACETALDEHYDE
ETHYL TRICYCLO[5.2.1.0.(2,6)]DECANE-2-CARBOXYLATE
(+)-(1′S,2S,E)-3,3-DIMETHYL-5-(2′,2′,3′-TRIMETHYL-3′-
CYCLOPENTEN-1′-YL)-4-PENTEN-2-OL
(2R,4E)-3,3-DIMETHYL-5-[(1R)-2,2,3-TRIMETHYL-3-
CYCLOPENTEN-1-YL]-4-PENTEN-2-OL (A) + (2S,4E)-3,3-
DIMETHYL-5-[(1R)-2,2,3-TRIMETHYL-3-CYCLOPENTEN-1-YL]-
4-PENTEN-2-OL (B)
8-ISOPROPYL-6-METHYL-BICYCLO[2.2.2]OCT-5-ENE-2-
CARBALDEHYDE
METHYLNONYLACETALDEHYDE
4-FORMYL-2-METHOXYPHENYL 2-METHYLPROPANOATE
(E)-4-DECENAL
(+−)-2-ETHYL-4-(2,2,3-TRIMETHYL-3-CYCLOPENTEN-1-YL)-2-
BUTEN-1-OL
(1R,5R)-4,7,7-TRIMETHYL-6-THIABICYCLO[3.2.1]OCT-3-ENE (A) +
(1R,4R,5R)-4,7,7-TRIMETHYL-6-THIABICYCLO[3.2.1]OCTANE
(B)
(−)-(3R)-3,7-DIMETHYL-1,6-OCTADIEN-3-OL
(E)-3-PHENYL-2-PROPENENITRILE
4-METHOXYBENZYL ACETATE
(E)-3-METHYL-5-(2,2,3-TRIMETHYL-3-CYCLOPENTEN-1-YL)-4-
PENTEN-2-OL
ALLYL (3-METHYLBUTOXY)ACETATE (A) + (+−)-ALLYL (2-
METHYLBUTOXY)ACETATE
(+−)-(2E)-1-(2,6,6-TRIMETHYL-2-CYCLOHEXEN-1-YL)-2-BUTEN-
1-ONE
(1E)-1-(2,6,6-TRIMETHYL-1-CYCLOHEXEN-1-YL)-1-PENTEN-3-
ONE

According to an embodiment, perfume raw materials having a Log T<-4 are chosen in the group consisting of aldehydes, ketones, alcohols, phenols, esters lactones, ethers, epoxydes, nitriles and mixtures thereof.

According to an embodiment, perfume raw materials having a Log T<-4 comprise at least one compound chosen in the group consisting of alcohols, phenols, esters lactones, ethers, epoxydes, nitriles and mixtures thereof, preferably in amount comprised between 20 and 70% by weight based on the total weight of the perfume raw materials having a Log T<-4.

According to an embodiment, perfume raw materials having a Log T<-4 comprise between 20 and 70% by weight of aldehydes, ketones, and mixtures thereof based on the total weight of the perfume raw materials having a Log T<-4.

The remaining perfume raw materials contained in the oil-based core may have therefore a Log T>-4.

Non limiting examples of perfume raw materials having a Log T>-4 are listed in table B below.

TABLE B
perfume raw materials having a Log T > −4
Perfume raw materials (Log T > −4)
ETHYL 2-METHYLBUTYRATE
(E)-3-PHENYL-2-PROPENYL
ACETATE
(+ −)-8-SEC-BUTYLQUINOLINE (A) +
(+ −)-6-SEC-BUTYLQUINOLINE
(+ −)-3-(1,3-BENZODIOXOL-5-YL)-2-
METHYLPROPANAL
VERDYLE PROPIONATE
1-(OCTAHYDRO-2,3,8,8-
TETRAMETHYL-2-NAPHTALENYL)-
1-ETHANONE
METHYL 2-((1RS,2RS)-3-OXO-2-
PENTYLCYCLOPENTYL)ACETATE
(+ −)-(E)-4-METHYL-3-DECEN-5-OL
2,4-DIMETHYL-3-CYCLOHEXENE-1-
CARBALDEHYDE
1,3,3-TRIMETHYL-2-
OXABICYCLO[2.2.2]OCTANE
TETRAHYDRO-4-METHYL-2-(2-
METHYL-1-PROPENYL)-2H-PYRAN
ALDEHYDE C 12
1-OXA-12-CYCLOHEXADECEN-2-
ONE (A) + 1-OXA-13-
CYCLOHEXADECEN-2-ONE (B)
(+ −)-3-(4-ISOPROPYLPHENYL)-2-
METHYLPROPANAL
ALDEHYDE C 11 LENIQUE
(+ −)-2,6-DIMETHYL-7-OCTEN-2-OL
(+ −)-2,6-DIMETHYL-7-OCTEN-2-OL
ALLYL 3-
CYCLOHEXYLPROPANOATE
(Z)-3-HEXENYL ACETATE
(2RS,5SR)-5-METHYL-2-(2-
PROPANYL)CYCLOHEXANONE (A) +
(2RS,5RS)-5-METHYL-2-(2-
PROPANYL)CYCLOHEXANONE (B)
ALLYL HEPTANOATE
(1RS,2RS)-2-(2-METHYL-2-
PROPANYL)CYCLOHEXYL ACETATE
(A) + (1RS,2SR)-2-(2-METHYL-2-
PROPANYL)CYCLOHEXYL ACETATE (B)
1,1-DIMETHYL-2-PHENYLETHYL
BUTYRATE
GERANYL ACETATE (A) + NERYL
ACETATE (B)
(+ −)-1-PHENYLETHYL ACETATE
1,1-DIMETHYL-2-PHENYLETHYL
ACETATE
3-METHYL-2-BUTENYL ACETATE
ETHYL 3-OXOBUTANOATE (A) <=>
(2Z)-ETHYL 3-HYDROXY-2-
BUTENOATE (B)
8-P-MENTHANOL
8-P-MENTHANYL ACETATE (A) +
1-P-MENTHANYL ACETATE (B)
(+ −)-2-(4-METHYL-3-CYCLOHEXEN-1-
YL)-2-PROPANYL ACETATE
(+ −)-2-METHYLBUTYL BUTANOATE
2-{(1S)-1-](1R)-3,3-
DIMETHYLCYCLOHEXYL]ETHOXY}-
2-OXOETHYL PROPIONATE
3,5,6-TRIMETHYL-3-CYCLOHEXENE-
1-CARBALDEHYDE (A) + 2,4,6-
TRIMETHYL-3-CYCLOHEXENE-1-
CARBALDEHYDE (B)
2-CYCLOHEXYLETHYL ACETATE
ALDEHYDE C 8
ETHYL BUTANOATE
(+ −)-(3E)-4-(2,6,6-TRIMETHYL-2-
CYCLOHEXEN-1-YL)-3-BUTEN-2-
ONE (A) + (3E)-4-(2,6,6-TRIMETHYL-
1-CYCLOHEXEN-1-YL)-3-BUTEN-2-
ONE (B);
1-[(1RS,6SR)-2,2,6-
TRIMETHYLCYCLOHEXYL]-3-
HEXANOL
1,3,3-TRIMETHYL-2-
OXABICYCLO[2.2.2]OCTANE
1,3,3-TRIMETHYL-2-
OXABICYCLO[2.2.2]OCTANE
ETHYL HEXANOATE
UNDECANAL
ALDEHYDE C 10
2-PHENYLETHYL ACETATE
(1S,2S,4S)-1,7,7-
TRIMETHYLBICYCLO[2.2.1]HEPTAN-
2-OL (A) + (1S,2R,4S)-1,7,7-
TRIMETHYLBICYCLO[2.2.1]HEPTAN-
2-OL (B)
(+ −)-3,7-DIMETHYL-3-OCTANOL
1-METHYL-4-(2-
PROPANYLIDENE)CYCLOHEXENE
(+)-(R)-4-(2-METHOXYPROPAN-2-
YL)-1-METHYLCYCLOHEX-1-ENE
VERDYL ACETATE
(3R)-1-[(1R,6S)-2,2,6-
TRIMETHYLCYCLOHEXYL]-3-
HEXANOL (A) + (3S)-1-[(1R,6S)-2,2,6-
TRIMETHYLCYCLOHEXYL]-3-
HEXANOL (B) + (3R)-1-[(1S,6S)-2,2,6-
TRIMETHYLCYCLOHEXYL[-3-
HEXANOL (C)
(+)-(1S,1′R)-2-[1-(3′,3′-DIMETHYL-1′-
CYCLOHEXYL)ETHOXY]-2-
METHYLPROPYL PROPANOATE

According to an embodiment, the oil phase (or the oil-based core) comprises 2-75 wt % of a density balancing material having a density greater than 1.07 g/cm³ and 25-98 wt % of a perfume oil comprising at least 15 wt % of high impact perfume raw materials having a Log T<-4.

The density of a component is defined as the ratio between its mass and its volume (g/cm³).

Several methods are available to determine the density of a component.

One may refer for example to the ISO 298:1998 method to measure d20 densities of essential oils.

According to an embodiment, the density balancing material is chosen in the group consisting of benzyl salicylate, benzyl benzoate, cyclohexyl salicylate, benzyl phenylacetate, phenylethyl phenoxyacetate, triacetin, methyl and ethyl salicylate, benzyl cinnamate, and mixtures thereof.

According to a particular embodiment, the density balancing material is chosen in the group consisting of benzyl salicylate, benzyl benzoate, cyclohexyl salicylate and mixtures thereof.

According to a particular embodiment, the hydrophobic material is free of any active ingredient (such as perfume). According to this particular embodiment, it comprises, preferably consists of hydrophobic solvents, preferably chosen in the group consisting of isopropyl myristate, tryglycerides (e.g. Neobee® MCT oil, vegetable oils), D-limonene, silicone oil, mineral oil, and mixtures thereof with optionally hydrophilic solvents preferably chosen in the group consisting of 1,4 butanediol, benzyl alcohol, triethyl citrate, triacetin, benzyl acetate, ethyl acetate, propylene glycol (1,2-propanediol), 1,3-Propanediol, dipropylene glycol, glycerol, glycol ethers and mixtures thereof.

By “flavour ingredient or composition” it is meant here a flavouring ingredient or a mixture of flavouring ingredients, solvent or adjuvants of current use for the preparation of a flavouring formulation, i.e. a particular mixture of ingredients which is intended to be added to an edible composition or chewable product to impart, improve or modify its organoleptic properties, in particular its flavour and/or taste. Taste modulator as also encompassed in said definition. Flavouring ingredients are well known to a skilled person in the art and their nature does not warrant a detailed description here, which in any case would not be exhaustive, the skilled flavourist being able to select them on the basis of his general knowledge and according to the intended use or application and the organoleptic effect it is desired to achieve. Many of these flavouring ingredients are listed in reference texts such as in the book by S. Arctander, Perfume and Flavor Chemicals, 1969, Montclair, N.J., USA, or its more recent versions, or in other works of similar nature such as Fenaroli's Handbook of Flavor Ingredients, 1975, CRC Press or Synthetic Food Adjuncts, 1947, by M. B. Jacobs, can Nostrand Co., Inc. Solvents and adjuvants or current use for the preparation of a flavouring formulation are also well known in the art.

In a particular embodiment, the flavour is selected from the group consisting of terpenic flavours including citrus and mint oil, and sulfury flavours.

According to any one of the invention's embodiment, the oil represents between about 10% and 60% w/w, or even between 20% and 50% w/w, by weight, relative to the total weight of the oil-in water emulsion.

### Optional Polyfunctional Monomer (Oil Phase)

According to an embodiment, a polyfunctional monomer is further added into the oil phase in addition to the hydrophobic material to reinforce the shell.

The polyfunctional monomer may be chosen in the group consisting of at least one polyisocyanate, poly maleic anhydride, poly acyl chloride, polyepoxide, acrylate monomers and polyalkoxysilane.

The polyfunctional monomer used in the process according to the invention may be present in amounts representing from 0.025% to 15%, preferably from 0.1 to 15%, more preferably from 0.1 to 6%, and even more preferably from 0.1 to 1% by weight of the slurry of step iv).

According to a particular embodiment, the polyfunctional monomer is at least one polyisocyanate having at least two isocyanate functional groups.

Suitable polyisocyanates used according to the invention include aromatic polyisocyanate, aliphatic polyisocyanate and mixtures thereof. Said polyisocyanate comprises at least 2, preferably at least 3 but may comprise up to 6, or even only 4, isocyanate functional groups. According to a particular embodiment, a triisocyanate (3 isocyanate functional group) is used.

According to one embodiment, said polyisocyanate is an aromatic polyisocyanate.

The term “aromatic polyisocyanate” is meant here as encompassing any polyisocyanate comprising an aromatic moiety. Preferably, it comprises a phenyl, a toluyl, a xylyl, a naphthyl or a diphenyl moiety, more preferably a toluyl or a xylyl moiety. Preferred aromatic polyisocyanates are biurets, polyisocyanurates and trimethylol propane adducts of diisocyanates, more preferably comprising one of the above-cited specific aromatic moieties. More preferably, the aromatic polyisocyanate is a polyisocyanurate of toluene diisocyanate (commercially available from Bayer under the tradename Desmodur® RC), a trimethylol propane-adduct of toluene diisocyanate (commercially available from Bayer under the tradename Desmodur® L75), a trimethylol propane-adduct of xylylene diisocyanate (commercially available from Mitsui Chemicals under the tradename Takenate® D-110N). In a most preferred embodiment, the aromatic polyisocyanate is a trimethylol propane-adduct of xylylene diisocyanate.

According to another embodiment, said polyisocyanate is an aliphatic polyisocyanate. The term “aliphatic polyisocyanate” is defined as a polyisocyanate which does not comprise any aromatic moiety. Preferred aliphatic polyisocyanates are a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100), among which a biuret of hexamethylene diisocyanate is even more preferred.

According to another embodiment, the at least one polyisocyanate is in the form of a mixture of at least one aliphatic polyisocyanate and of at least one aromatic polyisocyanate, both comprising at least two or three isocyanate functional groups, such as a mixture of a biuret of hexamethylene diisocyanate with a trimethylol propane-adduct of xylylene diisocyanate, a mixture of a biuret of hexamethylene diisocyanate with a polyisocyanurate of toluene diisocyanate and a mixture of a biuret of hexamethylene diisocyanate with a trimethylol propane-adduct of toluene diisocyanate. Most preferably, it is a mixture of a biuret of hexamethylene diisocyanate with a trimethylol propane-adduct of xylylene diisocyanate. Preferably, when used as a mixture the molar ratio between the aliphatic polyisocyanate and the aromatic polyisocyanate is ranging from 80:20 to 10:90.

According to this embodiment, an inner shell made of a polymerized polyfunctional monomer is formed by interfacial polymerization during the process. The formation of said inner shell can take place before, during or after the formation of the biopolymer shell.

According to a particular embodiment, the oil phase is free from polyisocyanate, preferably free from any polyfunctional monomer.

### Protein (Aqueous Phase)

The protein in the aqueous phase is used as an emulsifier and allows the stabilization of the oil droplets therein.

According to an embodiment, the protein is chosen in the group consisting of milk proteins, caseinate salts such as sodium caseinate or calcium caseinate, casein, whey protein, hydrolyzed proteins, gelatins, gluten, pea protein, soy protein, silk protein and mixtures thereof.

According to a particular embodiment, the protein comprises sodium caseinate.

The protein may be used in an amount comprised between 0.5 and 10%, preferably between 1 and 8%, more preferably between 2 and 4% by weight based on the total weight of the slurry as defined in step iv).

According to another particular embodiment, the protein is a mixture comprising sodium caseinate and at least one globular protein.

By “globular” protein, it should be understood a spherical protein characterized by a tertiary structure in the native state, and able to unfold and aggregate under the action of heat, pressure or specific chemicals.

As non-limiting examples of globular protein that can be used in the invention, one may cite whey protein, beta-lactoglobulin, ovalbumine, bovine serum albumin, vegetable proteins, and mixtures thereof.

According to a particular embodiment, the protein is a mixture comprising sodium caseinate and whey protein, preferably a mixture consisting of sodium caseinate and whey protein.

The weight ratio between sodium caseinate and the globular protein, preferably whey protein is comprised between 0.01 and 100, preferably between 0.1 and 10, more preferably between 0.2 and 5.

When the protein comprises a globular protein, the process preferably comprises a further heating step to denature the protein. Typically, the heating step is performed after the cross-linking step at a temperature comprised between 70° C. and 90° C.

Indeed, it has been found that the combination of an enzymatic cross-linking and a thermal annealing improves the performance of invention's microcapsules.

According to a particular embodiment, the process comprises the steps of:
(i) Admixing a salt into an aqueous solution comprising at least a protein to form an aqueous phase; wherein the protein is a mixture of sodium caseinate and whey protein
(ii) Dispersing an oil phase comprising a hydrophobic material, preferably a perfume oil or a flavor oil, and optionally a polyfunctional monomer into the aqueous phase to form an oil-in-water emulsion;
(iii) Adding into the oil-in-water emulsion an enzymatic cross-linker; preferably transglutaminase;
(iv) Applying sufficient conditions to induce the cross-linking of sodium caseinate by the cross-linker, and
(v) Applying sufficient conditions to induce the denaturation of whey protein, preferably by a heating treatment to form a biopolymer shell.

The heating step can be carried out at a temperature T_{den} (denaturation temperature of the protein), preferably comprised between 70° C. and 100° C., more preferably between 80° C. and 100° C. The duration of the heating step will depend on the heating temperature. Typically, the duration of the heating step is comprised between 10 and 60 minutes.

### Salt (Aqueous Phase)

According to the invention, the salt complexation of the protein is important for aggregation of the protein and maximizing the protein content at the oil/water interface.

The salt added in the aqueous phase can be chosen in the group consisting of calcium, sodium, potassium, lithium, magnesium, sulphates, phosphates, nitrates, bromides, chlorides, iodides, ammonium salts, and mixtures thereof.

According to an embodiment, the salt is chosen in the group consisting of CaCl₂), calcium acetate, calcium lactate, NaCl, KCl, LiCl, Ca(NO₃)₂, MgCl₂, CaBr₂, CaI₂, NaBr, NaI, NaNO₃, KBr, KI, KNO₃, LiBr, LiI, MgBr₂ and mixtures thereof.

According to an embodiment, the salt is chosen in the group consisting of CaCl₂), NaCl, KCl, LiCl, Ca(NO₃)₂, MgCl₂, and mixtures thereof.

When the process comprises a mineralization step, the salt is preferably chosen in the group consisting of calcium salts, preferably CaCl₂) or Ca(NO3)₂ as it is a precursor for the mineralization.

According to an embodiment, the weight ratio between the salt and the protein is comprised between 0.01:1 to 1:1, preferably between 0.1:1 and 0.4:1.

The emulsion may be prepared by high shear mixing and adjusted to the desired droplet size. The droplet size, comprised preferably between 1 and 1000 microns, more preferably between 10 and 50 microns, can be checked with light scattering measurements or microscopy. This procedure does not require a more detailed description here as it is well known to a skilled person in the art.

According to an embodiment, the mean droplet size is greater than 10 microns. According to an embodiment, the mean droplet size is greater than 20 microns.

According to an embodiment, the mean droplet size is comprised between 10 and 500 microns, preferably between 10 and 100 microns, more preferably between 10 and 50 microns.

According to an embodiment, the mean droplet size is comprised between 15 and 500 microns, preferably between 15 and 100 microns, more preferably between 15 and 50 microns.

According to an embodiment, the mean droplet size is comprised between 20 and 500 microns, preferably between 20 and 100 microns, more preferably between 20 and 50 microns.

### Cross-Linker

According to the invention, a cross-linker is added during the process to cross-link the protein.

The cross-linking is important for binding the protein together to form the biopolymer shell.

Even if the presence of the cross-linker is an essential feature of the present invention, said cross-linker can be added directly in the aqueous phase or, if not added in the aqueous phase, said cross-linker is added once the oil-in-water emulsion is formed.

The cross-linker can be added in step (i) in the aqueous phase and/or in step (iii) once the oil-in-water emulsion is formed.

According to a particular embodiment, the cross-linker is added once the oil-in-water emulsion is formed.

The cross-linker used in the present invention can be an enzymatic cross-linker such as an enzyme or a non-enzymatic cross-linker such as glutaraldehyde or genipin.

According to a particular embodiment, the cross-linker is an enzyme.

According to a particular embodiment, the enzyme is transglutaminase.

The enzyme may be used in an amount comprised between 0.001 and 0.1%, preferably between 0.005 and 0.02% based on the total weight of the slurry of step iii).

In some commercial products, the enzyme is dispersed in a carrier. One may cite for example Activa® TI (Origin: Ajinomoto). In other words, the commercial product is added in the process so as to have the enzyme actives in an amount preferably between 0.001 to 5%, preferably from 0.001 to 1%, even more preferably 0.001 and 0.1%, and even more preferably preferably between 0.005 and 0.02% based on the protein content and total weight of the slurry of step iii).

Action required to induce the cross-linking of the protein by the cross-linker is well known by the skilled person in the art. Typically, the oil-in-water emulsion comprising the cross-linker, preferably the enzyme is mixed at a temperature comprised between 35° C. and 55° C. for a time between 30 min and 4 hours to form the biopolymer shell.

When the cross-linker is an enzyme, once the biopolymer shell is formed, a heating treatment can be performed on the slurry to deactivate the enzyme. Typically, the heating treatment is performed at a temperature comprised between 70° C. and 90° C.

### Optional Heating Step

According to an embodiment, the process further comprises after the cross-linking step, a heating step, performed preferably at a temperature comprised between 70 and 90° C. This heating step can be used to deactivate the enzyme when the enzyme is used for the cross-linking and/or to induce the interfacial polymerization when a polyfunctional monomer is added in the oil phase and/or to induce the denaturation of the globular protein when the protein comprises a mixture of a non-globular protein with a globular protein (for example mixture of sodium caseinate and whey protein).

This heating step can also be used to further potentially bond materials, reduce interstitial spacing and thermally anneal the membrane to reduce defects and porosity.

Optional biomineralization step According to an embodiment, the process comprises after cross-linking step (iv) further steps consisting of
(v) optionally, adsorption of at least one mineral precursor on the microcapsule shell;
(vi) applying conditions suitable to induce growth of a mineral layer on the microcapsule shell.
   Additional step (v) can be omitted when the salt added in step (i) is the mineral precursor (for example when calcium chloride is used as a salt). In that case, the mineral precursor is throughout the membrane and not only at the surface.
   In other words, the mineral precursor might already be present from the salt-induced packing of proteins during and/or after emulsification.

Depending on the nature of the mineral precursor, prior to step (v), microcapsules may be concentrated or rinsed to remove the excess emulsifier solution. Microcapsules can be rinsed for example by centrifugation and resuspended in water after withdrawing the supernatant. This embodiment is particularly suitable when the mineral precursor solution is chosen in the group consisting of an iron (II) sulfate solution, or an iron (III) chloride solution.

Without being bound by theory, it is believed that the charged surface of the shell is providing functional anchoring sites and a high local density of charge groups and nucleation sites onto the surface of the microcapsules resulting in improved adsorption or absorption of mineral precursor species followed by initiation of the mineral growth process through in-situ addition of a precipitating species.

Mineral precursors are adsorbed to the surface of microcapsules by incubating the charged capsules in at least one solution containing oppositely charged mineral precursor, providing sufficient agitation and time to allow for complete coverage of capsule surfaces. Removal of excess precursor from solution to prevent generation of free mineral material in solution can be done and is followed by initiation of the mineral growth process through in-situ addition of a precipitating species. Removal of excess precursor is not necessary in all embodiments, especially when mineral growth is achieved slowly by reacting low concentrations of mineral precursors to selectively grow material onto the biopolymer shell.

The person skilled in the art will be able to select suitable conditions for the mineral growth process (for example, precursor selection, reaction conditions, the solution concentrations, incubation times, agitation speeds, temperatures and pH conditions).

### Typically:

mineralization may occur at room temperature,
mineralization process may begin following the addition of the mineral precursor or following the addition of a precipitation species (after addition of the mineral precursor)
depending on the nature of the mineral precursor, process duration my vary from 1 to 24 hours.

According to a particular embodiment, the mineral precursor solution is chosen in the group consisting of an iron (II) sulphate solution (comprising iron ions as precursor), an iron (III) chloride solution (comprising iron ions as precursor), calcium-based salt solution (comprising calcium ions as precursor), phosphate-based salt solution (comprising phosphate ions as precursor), carbonate-based salt solution (comprising carbonate ions as precursor), titanium-based precursor solution, zinc-based precursor solution, and mixtures thereof.

One may cite for example titanium alkoxides as titanium-based precursor or zinc alkoxides, zinc acetate, zinc chloride as zinc-based precursor solution.

According to a particular embodiment, the mineral precursor solution is chosen in the group consisting of an iron (II) sulfate solution (comprising iron ions as precursor), an iron (III) chloride solution (comprising iron ions as precursor), calcium-based salt solution (comprising calcium ions as precursor), phosphate-based salt solution (comprising phosphate ions as precursor) and mixtures thereof.

The water-soluble calcium-based salt can be chosen in the group consisting of calcium chloride (CaCl₂)), calcium nitrate (Ca(NO₃)₂), calcium bromide (CaBr₂), calcium iodide (CaI₂), calcium chromate (CaCrO₄), calcium acetate (CaCH₃CO₂) and mixtures thereof. Most preferred are calcium chloride and calcium nitrate.

The water-soluble phosphate-based salt can be chosen in the group consisting of sodium phosphate (monobasic) (NaH₂PO₄), sodium phosphate (dibasic) (Na₂HPO₄), sodium phosphate (tribasic): Na₃PO₄, potassium phosphate (monobasic): KH₂PO₄, potassium phosphate (dibasic) (K₂HPO₄), potassium phosphate (tribasic) (K₃PO₄), ammonium phosphate (monobasic) ((NH₄)H₂PO₄), ammonium phosphate(dibasic) ((NH₄)₂HPO₄), ammonium phosphate(tribasic) ((NH₄)₃PO₄) and mixtures thereof.

The water-soluble carbonate-based salt can be chosen in the group consisting of sodium, potassium and ammonium based carbonates.

It should be understood that the charge of the mineral precursor used in step (v) of the process is driven by the charge of the terminating surface of the microcapsules, the solution conditions (including pH) and the affinity of the terminating surface for the mineral precursor.

After step (iv), the biopolymer shell is preferably negatively charged.

However, the surface of the biopolymer shell can be modified with alternating polyelectrolyte layers or adsorption of a functional coating prior to adsorption of the mineral precursor.

This embodiment is not limited to only one layer or one pair of opposite polyelectrolyte layers but includes 2, 3, 4 or even more of layers or pair of opposite polyelectrolyte layers. The charge and functionality of the last layer determines the charge and functionality of the mineral precursor added in step (v).

According to an embodiment, the cationic polyelectrolyte layer is chosen in the group consisting of poly(allylamine hydrochloride), poly-L-lysine and chitosan.

According to another embodiment, the anionic polyelectrolyte layer is chosen in the group consisting of poly(sodium 4 styrene sulfonate) (PSS), polyacrylic acid, polyethylene imine, humic acid, carrageenan, pectin, gum acacia, and mixtures thereof.

According to a particular embodiment, the anionic polyelectrolyte layer is PSS.

### Embodiment 1

According to an embodiment, the mineral precursor solution is chosen in the group consisting of an iron (II) sulfate solution, or an iron (III) chloride solution.

The initiation of the mineral growth process can be done through in-situ addition of a precipitating species. According to this embodiment, when the mineral precursor is an iron solution, iron ions are adsorbed on the anionic surface of the shell and precipitating species used is a base for hydrolysis to form an iron oxide layer (for example by addition of a sodium hydroxide solution).

The weight ratio between the mineral precursor salt in solution and the microcapsules slurry of step iv) can be comprised between 1:1 and 2:1, preferably between 1.3:1 and 1.7:1, and most preferably between 1.5:1 and 1.6:1. Values are given for pure salts in solution—the person skilled in the art will be able to adapt the amount of the salt if a hydrated form is used.

### Embodiment 2

According to an embodiment, the mineral precursor solution is chosen in the group consisting of sodium carbonate Na₂CO₃, calcium chloride CaCl₂), sodium phosphate dibasic Na₂HPO₄, sodium phosphate monobasic NaH₂PO₄, sodium phosphate tribasic Na₃PO₄, calcium nitrate Ca(NO₃)₂.

According to a particular embodiment when calcium chloride CaCl₂) or Ca(NO₃)₂ is used as a salt in step i) of the process, only the mineral precursor, namely Na₂CO₃ or NaH₂PO₄ can be added to form respectively a mineral layer made of calcium carbonate CaCO₃ or calcium phosphate CaPO₄.

However, to improve the robustness of the shell, microcapsules can be then incubated again several times simultaneously or sequentially in the two following precursor solutions (Na₂CO₃/CaCl₂) or NaH₂PO₄/CaCl₂)).

### Embodiment 3

According to this particular embodiment, microcapsules are introduced sequentially or simultaneously in at least two solutions comprising respectively at least one precursor. Preferably, the first solution comprises water-soluble calcium-based salt including a calcium precursor (first mineral precursor of step v)) and the second solution comprises water-soluble phosphate-based salt including a phosphate precursor (second mineral precursor to induce the growth of the mineral layer). Addition order could change according to the selection and charge of the underlying terminating layer.

According to a particular embodiment, the first solution comprises calcium nitrate (Ca(NO₃)₂) and the second solution comprises sodium phosphate (dibasic) (Na₂HPO₄).

According to another particular embodiment, the first solution comprises calcium chloride (CaCl₂)) and the second solution comprises sodium carbonate (Na₂CO₃).

To improve the robustness of the shell, microcapsules can be then incubated again several times simultaneously or sequentially in the two mineral precursor solutions.

### Embodiment 4

Still according to another embodiment, the microcapsules are firstly incubating in carbonate-based salt solution or in a phosphate-based salt solution to adsorb carbonate ions CO₃²⁻ or phosphate ions PO₄³⁻respectively on the surface followed by an incubation in a calcium-based mineral solution.

According to another embodiment, the first solution comprises a water-soluble carbonate-based salt including a carbonate precursor and the second solution comprises a water-soluble calcium-based salt including a calcium precursor.

More specifically, according to a particular embodiment, the first solution comprises sodium carbonate Na₂CO₃ and the second solution comprises calcium chloride CaCl₂).

To improve the robustness of the shell, microcapsules can be then incubated again several times simultaneously or sequentially in the two mineral precursor solutions.

According to different embodiments described above, the weight ratio between the first mineral precursor salts in solution and the microcapsules slurry of step iv) can be comprised between 0.01:1 and 0.5:1, more preferably between 0.03:1 and 0.4:1, and the weight ratio between the second mineral precursor solution and the microcapsules slurry of step iv) can be comprised between 0.01:1 and 0.5:1, preferably between 0.03:1 and 0.4:1.

According to a particular embodiment, the weight ratio between the first mineral precursor salts in solution and the microcapsules slurry of step iv) can be comprised between 0.1:1 and 0.5:1, preferably between 0.15:1 and 0.4:1, and the weight ratio between the second mineral precursor solution and the microcapsules slurry of step iv) can be comprised between 0.05:1 and 0.3:1, preferably between 0.08:1 and 0.25:1. Values are given for pure salts in solution—the person skilled in the art will be able to adapt the amount of the salt if a hydrated form is used.

According to the different embodiments described above, once a mineral layer is formed, one may repeat the biomineralization step with other mineral precursors so as to form at least a second mineral layer different from the first mineral layer. Polyelectrolyte layers can be formed between the mineral layers.

### Optional Outer Coating

According to a particular embodiment of the invention, during or at the end of step iv) and/or following the mineralization step, one may also add to the invention's slurry a polymer selected from the group consisting of a polysaccharide, a biopolymer, a cationic polymer and mixtures thereof to form an outer coating to the microcapsule.

Polysaccharide polymers are well known to a person skilled in the art. Preferred non-ionic polysaccharides are selected from the group consisting of locust bean gum, xyloglucan, guar gum, hydroxypropyl guar, hydroxypropyl cellulose and hydroxypropyl methyl cellulose, pectin and mixtures thereof.

According to a particular embodiment, the coating consists of a cationic coating.

Cationic polymers are also well known to a person skilled in the art. Preferred cationic polymers have cationic charge densities of at least 0.5 meq/g, more preferably at least about 1.5 meq/g, but also preferably less than about 7 meq/g, more preferably less than about 6.2 meq/g. The cationic charge density of the cationic polymers may be determined by the Kjeldahl method as described in the US Pharmacopoeia under chemical tests for Nitrogen determination. The preferred cationic polymers are chosen from those that contain units comprising primary, secondary, tertiary and/or quaternary amine groups that can either form part of the main polymer chain or can be borne by a side substituent directly connected thereto. The weight average (Mw) molecular weight of the cationic polymer is preferably between 10,000 and 3.5M Dalton, more preferably between 50,000 and 2M Dalton.

According to a particular embodiment, one will use cationic polymers based on acrylamide, methacrylamide, N-vinylpyrrolidone, quaternized N,N-dimethylaminomethacrylate, diallyldimethylammonium chloride, quaternized vinylimidazole (3-methyl-1-vinyl-1H-imidazol-3-ium chloride), vinylpyrrolidone, acrylamidopropyltrimonium chloride, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. Preferably copolymers shall be selected from the group consisting of polyquaternium-5, polyquaternium-6, polyquaternium-7, polyquaternium10, polyquaternium-11, polyquaternium-16, polyquaternium-22, polyquaternium-28, polyquaternium-43, polyquaternium-44, polyquaternium-46, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride

As specific examples of commercially available products, one may cite Salcare® SC60 (cationic copolymer of acrylamidopropyltrimonium chloride and acrylamide, origin: BASF) or Luviquat®, such as the PQ 1N, FC 550 or Style (polyquaternium-11 to 68 or quaternized copolymers of vinylpyrrolidone origin: BASF), or also the Jaguar@ (C13S or C17, origin Rhodia).

When the coating is added after the mineralization step, depending on the charge of the mineralized microcapsules surface, and solution conditions, an anionic polyelectrolyte can be first adsorbed on the surface followed by the adsorption of a cationic polymer. Or, a cationic polymer could be adsorbed followed by adsorption of an anionic coating.

Post-functionalization of the mineralized shell could be done to impart greater barrier functionality, to serve as a foundation for further enzymatic crosslinking, to serve as a foundation for further mineralization, or to offer a differently functionalized surface to facilitate compatibility with application bases or performance (such as deposition performance) from application bases.

According to any one of the above embodiments of the invention, there is added an amount of polymer described above comprised between about 0% and 5% w/w, or even between about 0.1% and 2% w/w, percentage being expressed on a w/w basis relative to the total weight of the slurry as obtained after step iv) or vi). It is clearly understood by a person skilled in the art that only part of said added polymers will be incorporated into/deposited on the microcapsule shell.

### Multiple Microcapsules System

According to an embodiment, the microcapsules of the invention (first microcapsule slurry) can be used in combination with a second microcapsules slurry.
Another object of the invention is a microcapsule delivery system comprising:
the microcapsule slurry of the present invention as a first microcapsule slurry, and
a second microcapsule slurry, wherein the microcapsules contained in the first microcapsule slurry and the second microcapsule slurry differ in their hydrophobic material and/or their wall material and/or in their coating material and/or in their mineral layer.

As non-limiting examples, the nature of the polymeric shell of microcapsules from the second microcapsules slurry of the invention can vary. As non-limiting examples, the shell of the second microcapsules slurry can be aminoplast-based, polyurea-based or polyurethane-based. The shell of the second microcapsules slurry can also be hybrid, namely organic-inorganic such as a hybrid shell composed of at least two types of inorganic particles that are cross-linked, or yet a shell resulting from the hydrolysis and condensation reaction of a polyalkoxysilane macro-monomeric composition.

According to an embodiment, the shell of the second microcapsules slurry comprises an aminoplast copolymer, such as melamine-formaldehyde or urea-formaldehyde or cross-linked melamine formaldehyde or melamine glyoxal.

According to another embodiment the shell of the second microcapsules slurry is polyurea-based made from, for example but not limited to isocyanate-based monomers and amine-containing crosslinkers such as guanidine carbonate and/or guanazole. Preferred polyurea microcapsules comprise a polyurea wall which is the reaction product of the polymerisation between at least one polyisocyanate comprising at least two isocyanate functional groups and at least one reactant selected from the group consisting of an amine (for example a water soluble guanidine salt and guanidine); a colloidal stabilizer or emulsifier; and an encapsulated perfume. However, the use of an amine can be omitted.

According to a particular embodiment the colloidal stabilizer includes an aqueous solution of between 0.1% and 0.4% of polyvinyl alcohol, between 0.6% and 1% of a cationic copolymer of vinylpyrrolidone and of a quaternized vinylimidazol (all percentages being defined by weight relative to the total weight of the colloidal stabilizer). According to another embodiment, the emulsifier is an anionic or amphiphilic biopolymer preferably chosen from the group consisting of gum Arabic, soy protein, gelatin, sodium caseinate and mixtures thereof.

According to another embodiment, the shell of the second microcapsules slurry is polyurethane-based made from, for example but not limited to polyisocyanate and polyols, polyamide, polyester, etc.

The preparation of an aqueous dispersion/slurry of core-shell microcapsules is well known by a skilled person in the art. In one aspect, said microcapsule wall material may comprise any suitable resin and especially including melamine, glyoxal, polyurea, polyurethane, polyamide, polyester, etc. Suitable resins include the reaction product of an aldehyde and an amine, suitable aldehydes include, formaldehyde and glyoxal. Suitable amines include melamine, urea, benzoguanamine, glycoluril, and mixtures thereof. Suitable melamines include, methylol melamine, methylated methylol melamine, imino melamine and mixtures thereof. Suitable ureas include, dimethylol urea, methylated dimethylol urea, urea-resorcinol, and mixtures thereof. Suitable materials for making may be obtained from one or more of the following companies Solutia Inc. (St Louis, Missouri U.S.A.), Cytec Industries (West Paterson, New Jersey U.S.A.), Sigma-Aldrich (St. Louis, Missouri U.S.A.).

According to a particular embodiment, the second core-shell microcapsule is a formaldehyde-free capsule. A typical process for the preparation of aminoplast formaldehyde-free microcapsules slurry comprises the steps of 1) preparing an oligomeric composition comprising the reaction product of, or obtainable by reacting together
a) a polyamine component in the form of melamine or of a mixture of melamine and at least one C₁-C₄ compound comprising two NH₂ functional groups;
b) an aldehyde component in the form of a mixture of glyoxal, a C₄₋₆ 2,2-dialkoxy-ethanal and optionally a glyoxalate, said mixture having a molar ratio glyoxal/C₄₋₆ 2,2-dialkoxy-ethanal comprised between 1/1 and 10/1; and
c) a protic acid catalyst;
   2) preparing an oil-in-water dispersion, wherein the droplet size is comprised between 1 and 600 um, and comprising:
i. an oil;
ii. a water medium
iii. at least an oligomeric composition as obtained in step 1;
iv. at least a cross-linker selected amongst
A) C₄-C₁₂ aromatic or aliphatic di- or tri-isocyanates and their biurets, triurets, trimmers, trimethylol propane-adduct and mixtures thereof, and/or
B) a di- or tri-oxiran compounds of formula
A-(oxiran-2-ylmethyl)ₙ
wherein n stands for 2 or 3 and 1 represents a C₂-C₆ group optionally comprising from 2 to 6 nitrogen and/or oxygen atoms;
   v. optionally a C₁-C₄ compounds comprising two NH₂ functional groups;
      3) Heating said dispersion;
      4) Cooling said dispersion.
      This process is described in more details in WO 2013/068255, the content of which is included by reference.

According to another embodiment, the shell of the of the second microcapsules slurry is polyurea-or polyurethane-based. Examples of processes for the preparation of polyurea and polyureathane-based microcapsule slurry are for instance described in WO2007/004166, EP 2300146, EP2579976 the contents of which is also included by reference. Typically a process for the preparation of polyurea or polyurethane-based microcapsule slurry include the following steps:
a) Dissolving at least one polyisocyanate having at least two isocyanate groups in an oil to form an oil phase;
b) Preparing an aqueous solution of an emulsifier or colloidal stabilizer to form a water phase;
c) Adding the oil phase to the water phase to form an oil-in-water dispersion, wherein the mean droplet size is comprised between 1 and 500 μm, preferably between 5 and 50 μm;
d) Applying conditions sufficient to induce interfacial polymerisation and form microcapsules in form of a slurry.

### Process for Preparing a Microcapsule Powder

Another object of the invention is a process for preparing a microcapsule powder comprising the steps as defined above and an additional step consisting of submitting the microcapsule slurry obtained in step iv) or vi) to a drying, like spray-drying, to provide the microcapsules as such, i.e. in a powdery form. It is understood that any standard method known by a person skilled in the art to perform such drying is also applicable. In particular the slurry may be spray-dried preferably in the presence of a polymeric carrier material such as polyvinyl acetate, polyvinyl alcohol, dextrins, natural or modified starch, gum Arabic, vegetable gums, pectins, xanthans, alginates, carrageenans or cellulose derivatives to provide microcapsules in a powder form.

According to a particular embodiment, the carrier material contains free perfume oil which can be the same or different from the perfume from the core of the microcapsules.

### Microcapsule Slurry/Microcapsule Powder

Microcapsule slurry and microcapsule powder obtainable by the processes above-described are also an object of the invention.

Another object of the present invention is a core-shell microcapsules slurry comprising at least one microcapsules made of:
an oil-based core
optionally an inner shell made of a polymerized polyfunctional monomer;
a biopolymer shell comprising a protein, wherein at least one protein is cross-linked; and
optionally at least an outer mineral layer.
   All the previous embodiments described previously for the process for preparing the microcapsule slurry also apply for the microcapsule slurry described above.
   The definitions of hydrophobic material, protein, the polyfunctional monomer, the outer mineral layer are the same as described hereinabove.
   According to the invention, the oil-based core comprises a hydrophobic material as defined previously.

According to an embodiment, the mineral layer comprises a material chosen in the group consisting of iron oxides, iron oxyhydroxide, titanium oxides, zinc oxides, calcium carbonates, calcium phosphates and mixtures thereof.

According to an embodiment, the mineral layer comprises a material chosen in the group consisting of iron oxides, iron oxyhydroxide, titanium oxides, zinc oxides, calcium carbonates, calcium phosphates and mixtures thereof. Preferably, the mineral layer is an iron oxide, an iron oxyhydroxide, or a calcium phosphate or a calcium carbonate. All crystalline minerals, amorphous minerals and mineral polymorphs (such as hydroxyapatite for calcium phosphate; and calcite, vaterite, and aragonite for calcium carbonate) are included.

According to a particular embodiment, the mineral layer is iron oxyhydroxide goethite (α-FeO(OH)).

According to another embodiment, the mineral layer is calcium phosphate.

According to another embodiment, the mineral layer is calcium carbonate.

According to another embodiment, multiple mineral layers comprising calcium phosphate and calcium carbonate are present.

According to a particular embodiment, the microcapsules comprise an outer coating as described previously on the biopolymer shell and/or on the optional mineral layer.

According to an embodiment, the protein is chosen in the group consisting of milk proteins, caseinate salts such as sodium caseinate or calcium caseinate, casein, whey protein, hydrolyzed proteins, gelatins, gluten, pea protein, soy protein, silk protein and mixtures thereof.

According to an embodiment, the protein(s) contained in the biopolymer shell consist of cross-linked protein(s).

According to an embodiment, the protein comprises sodium caseinate, preferably cross-linked sodium caseinate.

According to an embodiment, the protein comprises sodium caseinate and a globular protein, preferably chosen in the group consisting of whey protein, beta-lactoglobulin, ovalbumine, bovine serum albumin, vegetable proteins, and mixtures thereof.

The protein is preferably a mixture of sodium caseinate and whey protein.

According to an embodiment, the biopolymer shell comprises a crosslinked protein chosen in the group consisting of sodium caseinate and/or whey protein.

According to a particular embodiment, the microcapsules slurry comprises at least one microcapsule made of:
an oil-based core, preferably comprising a perfume oil
an inner shell made of a polymerized polyfunctional monomer; preferably a polyisocyanate having at least two isocyanate functional groups
a biopolymer shell comprising a protein, wherein at least one protein is cross-linked; wherein the protein contains preferably a mixture comprising sodium caseinate and a globular protein, preferably whey protein.
optionally at least an outer mineral layer.

According to an embodiment, sodium caseinate and/or whey protein is (are) cross-linked protein(s).

The weight ratio between sodium caseinate and whey protein is preferably comprised between 0.01 and 100, preferably between 0.1 and 10, more preferably between 0.2 and 5.

According to another particular embodiment, the microcapsules slurry comprises at least one microcapsule made of:
an oil-based core, preferably comprising a perfume oil
a biopolymer shell comprising a protein, wherein at least one protein is cross-linked;
wherein the protein is preferably a mixture comprising sodium caseinate and whey protein,
optionally at least an outer mineral layer,
wherein the shell is free from polyisocyanate, preferably free from any polymerized polyfunctional monomer.

The biopolymer shell may comprise a salt and a cross-linker as defined previously.

It has to be mentioned that although ideal situation would be one where microcapsules show best stability, i.e. lowest active leakage in application combined with best delivery performance, such as perfume intensity in the case of a perfume in application both before rubbing and after rubbing, different scenarios can be very interesting depending on the application and slightly less stable capsules with higher odor performance can be very useful and so could more stable capsules with slightly lower odor performance. A skilled person in the art is capable of choosing the best balance depending on the needs in application.

### Consumer Products

The microcapsules of the invention can be used in combination with active ingredients. An object of the invention is therefore a composition comprising:
(i) microcapsules as defined above;
(ii) an active ingredient, preferably chosen in the group consisting of a cosmetic ingredient, skin caring ingredient, perfume ingredient, flavor ingredient, malodour counteracting ingredient, bactericide ingredient, fungicide ingredient, pharmaceutical or agrochemical ingredient, a sanitizing ingredient, an insect repellent or attractant, and mixtures thereof.

The microcapsules of the invention can be used for the preparation of perfuming or flavouring compositions which are also an object of the invention.

### Perfumed Consumer Products

The microcapsules of the invention can also be added in different perfumed consumer products.

In particular a perfuming composition comprising (i) microcapsules as defined above; (ii) at least one perfuming co-ingredient; and (iii) optionally a perfumery adjuvant, is another object of the invention.

By “perfuming co-ingredient” it is meant here a compound, which is used in a perfuming preparation or a composition to impart a hedonic effect and which is not a microcapsule as defined above. In other words such a co-ingredient, to be considered as being a perfuming one, must be recognized by a person skilled in the art as being able to impart or modify in a positive or pleasant way the odor of a composition, and not just as having an odor. The nature and type of the perfuming co-ingredients present in the perfuming composition do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said co-ingredients may also be compounds known to release in a controlled manner various types of perfuming compounds.

By “perfumery adjuvant” we mean here an ingredient capable of imparting additional added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

Preferably, the perfuming composition according to the invention comprises between 0.1 and 30% by weight of microcapsules as defined above.

The invention's microcapsules can advantageously be used in many application fields and used in consumer products. Microcapsules can be used in liquid form applicable to liquid consumer products as well as in powder form, applicable to powder consumer products.

In the case of microcapsules including a perfume oil-based core, the products of the invention, can in particular be of use in perfumed consumer products such as product belonging to fine fragrance or “functional” perfumery. Functional perfumery includes in particular personal-care products including hair-care, body cleansing, skin care, hygiene-care as well as home-care products including laundry care and air care. Consequently, another object of the present invention consists of a perfumed consumer product comprising as a perfuming ingredient, the microcapsules defined above or a perfuming composition as defined above. The perfume element of said consumer product can be a combination of perfume microcapsules as defined above and free or non-encapsulated perfume, as well as other types of perfume microcapsule than those here-disclosed.

In particular a liquid consumer product comprising:
from 2 to 65% by weight, relative to the total weight of the consumer product, of at least one surfactant;
water or a water-miscible hydrophilic organic solvent; and
a perfuming composition or microcapsules as defined above, wherein the active ingredient comprise a perfume is another object of the invention.

Also a powder consumer product comprising
from 2 to 65% by weight, relative to the total weight of the consumer product, of at least one surfactant; and
a perfuming composition or microcapsules, wherein the active ingredient comprise a perfume as defined above is part of the invention.

According to a particular embodiment, the process for preparing the microcapsules contained in the perfumed consumer product comprises the addition of a polyisocyanate into the oil phase to improve the stability in challenging bases comprising a high amount of surfactants.

The invention's microcapsules can therefore be added as such or as part of an invention's perfuming composition in a perfumed consumer product.

For the sake of clarity, it has to be mentioned that, by “perfumed consumer product” it is meant a consumer product which is expected to deliver among different benefits a perfuming effect to the surface to which it is applied (e.g. skin, hair, textile, paper, or home surface) or in the air (air-freshener, deodorizer etc). In other words, a perfumed consumer product according to the invention is a manufactured product which comprises a functional formulation also referred to as “base”, together with benefit agents, among which an effective amount of microcapsules according to the invention.

The nature and type of the other constituents of the perfumed consumer product do not warrant a more detailed description here, which in any case would not be exhaustive, the skilled person being able to select them on the basis of his general knowledge and according to the nature and the desired effect of said product. Base formulations of consumer products in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Non-limiting examples of suitable perfumed consumer product can be a perfume, such as a fine perfume, a cologne, an after-shave lotion, a body-splash; a fabric care product, such as a liquid or solid detergent, tablets and pods, a fabric softener, a dryer sheet, a fabric refresher, an ironing water, or a bleach; a personal-care product, such as a hair-care product (e.g. a shampoo, hair conditioner, a colouring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product); an air care product, such as an air freshener or a “ready to use” powdered air freshener; or a home care product, such all-purpose cleaners, liquid or powder or tablet dishwashing products, toilet cleaners or products for cleaning various surfaces, for example sprays & wipes intended for the treatment/refreshment of textiles or hard surfaces (floors, tiles, stone-floors etc.); a hygiene product such as sanitary napkins, diapers, toilet paper.

Another object of the invention is a consumer product comprising:
a personal care active base, and
microcapsules as defined above or the perfuming composition as defined above,
   wherein the consumer product is in the form of a personal care composition.

Personal care active base in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

The personal care composition is preferably chosen in the group consisting of a hair-care product (e.g. a shampoo, hair conditioner, a colouring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product) or a fine fragrance product (e.g. Eau de Toilette—EdT).

Another object of the invention is a consumer product comprising:
a home care or a fabric care active base, and
microcapsules as defined above or the perfuming composition as defined above,
   wherein the consumer product is in the form of a home care or a fabric care composition.
   Home care or fabric care bases in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature. The home or fabric care composition is preferably chosen in the group consisting fabric softener, liquid detergent, powder detergent, liquid scent booster solid scent booster.

According to a particular embodiment, the consumer product is in the form of a fabric softener composition and comprises:
between 85 and 99.9% of a fabric softener active base;
between 0.1 to 15 wt %, more preferably between 0.2 and 5 wt % by weight of the microcapsule slurry of the invention.
The fabric softener active base may comprise cationic surfactants of quaternary ammonium, such as Diethyl ester dimethyl ammonium chloride (DEEDMAC), TEAQ (triethanolamine quat), HEQ (Hamburg esterquat).
   According to a particular embodiment, the consumer product is in the form of a perfuming composition comprising:
0.1 to 20% of microcapsules as defined previously,
0 to 40%, preferably 3-40% of perfume, and
20-90, preferably 40-90% of ethanol, by weight based on the total weight of the perfuming composition.

Preferably, the consumer product comprises from 0.1 to 15 wt %, more preferably between 0.2 and 5 wt % of the microcapsules of the present invention, these percentages being defined by weight relative to the total weight of the consumer product. Of course the above concentrations may be adapted according to the benefit effect desired in each product.

Flavored Consumer Products

The microcapsules of the invention when encapsulating a flavour, can be used in a great variety of edible end products. Consumer products susceptible of being flavoured by the microcapsules of the invention may include foods, beverages, pharmaceutical and the like. For example foodstuff base that could use the slurries or powdered microcapsules of the invention include
Baked goods (e.g. bread, dry biscuits, cakes, other baked goods),
Non-alcoholic beverages (e.g. carbonated soft drinks, bottled waters, sports/energy drinks, juice drinks, vegetable juices, vegetable juice preparations),
Alcoholic beverages (e.g. beer and malt beverages, spirituous beverages),
Instant beverages (e.g. instant vegetable drinks, powdered soft drinks, instant coffee and tea),
Cereal products (e.g. breakfast cereals, pre-cooked ready-made rice products, rice flour products, millet and sorghum products, raw or pre-cooked noodles and pasta products),
Milk products (e.g. fresh cheese, soft cheese, hard cheese, milk drinks, whey, butter, partially or wholly hydrolysed milk protein-containing products, fermented milk products, condensed milk and analogues),
Dairy based products (e.g. fruit or flavored yoghurt, ice cream, fruit ices)
Confectionary products (e.g. chewing gum, hard and soft candy)
Chocolate and compound coatings
Products based on fat and oil or emulsions thereof (e.g. mayonnaise, spreads, margarines, shortenings, remoulade, dressings, spice preparations),
Spiced, marinated or processed fish products (e.g. fish sausage, surimi),
Eggs or egg products (dried egg, egg white, egg yolk, custard),
Desserts (e.g. gelatins and puddings)
Products made of soya protein or other soya bean fractions (e.g. soya milk and products made therefrom, soya lecithin-containing preparations, fermented products such as tofu or tempeh or products manufactured therefrom, soya sauces),
Vegetable preparations (e.g. ketchup, sauces, processed and reconstituted vegetables, dried vegetables, deep frozen vegetables, pre-cooked vegetables, vegetables pickled in vinegar, vegetable concentrates or pastes, cooked vegetables, potato preparations),
Vegetarian meat replacer, vegetarian burger
Spices or spice preparations (e.g. mustard preparations, horseradish preparations), spice mixtures and, in particular seasonings which are used, for example, in the field of snacks.
Snack articles (e.g. baked or fried potato crisps or potato dough products, bread dough products, extrudates based on maize, rice or ground nuts),
Meat products (e.g. processed meat, poultry, beef, pork, ham, fresh sausage or raw meat preparations, spiced or marinated fresh meat or cured meat products, reformed meat),
Ready dishes (e.g. instant noodles, rice, pasta, pizza, tortillas, wraps) and soups and broths (e.g. stock, savory cube, dried soups, instant soups, pre-cooked soups, retorted soups), sauces (instant sauces, dried sauces, ready-made sauces, gravies, sweet sauces).
Oral care products (toothpastes, tooth powders, flavored dental flosses, mouth washes . . . )
   Preferably, the microcapsules according to the invention shall be used in products selected from the group consisting of baked goods, instant beverages, cereal products, milk products, dairy-based products, products based on fat and oil or emulsions thereof, desserts, vegetable preparations, vegetarian meat replacer, spices and seasonings, snacks, meat products, ready dishes, soups and broths and sauces.

The invention will now be further described by way of examples. It will be appreciated that the invention as claimed is not intended to be limited in any way by these examples.

### EXAMPLES

### Example 1

### Preparation of Microcapsules by the Process of the Invention (Calcium Carbonate as a Mineral Layer)

Microcapsules A-D were prepared according to the following protocol.
1) Sodium caseinate is dissolved in DI water at RT.
2) Calcium chloride (aqueous solution) is slowly added to the sodium caseinate solution and stirred at RT for ˜15 min.
3) The emulsifier solution is combined with a perfume oil (see table 1) containing a polyisocyanate (Takenate® D-110N) and homogenized (18,000 rpm for 3 min).
4) The emulsion is then transferred to a reactor, pH adjusted to ˜6.5 w/NaOH, and heated to 45° C.
5) Transglutaminase (aqueous solution) is added to the reactor and it is stirred for 3 hr at 45° C.
6) The reactor is then heated to 70° C. and held for 30 min before cooling to RT.
   Some microcapsules were further mineralized with calcium carbonate (CaCO₃) by adding Na₂CO₃/CaCl₂) respectively according to the following protocol.
1) Added 20 g of the microcapsules slurry to 180 g of DI water and stirred at room temperature (250 rpm, 25° C.)
2) Slowly added 13.6 mL of 0.1M Na₂CO₃ over 1 hr (0.23 mL/min) and then stirred for 1 hr
3) Slowly added 13.6 mL of 0.1M CaCl₂) over 1 hr (0.23 mL/min) and then stirred for 1 hr
4) Repeated additions of Na₂CO₃ and CaCl₂) 3 more times (4 cycles total)

TABLE 1
Perfume oil A composition

| Ingredient | Parts |
|---|---|
| Isopropyl myristate | 0.3 |
| (Z)-3-hexen-1-ol butyrate | 0.6 |
| Delta damascene | 1.0 |
| 2,4-Dimethyl-3-cyclohexene- | 1.0 |
| 1-carbaldehyde | |
| Habanolide ®¹⁾ | 3.0 |
| Hedione ®²⁾ | 5.0 |
| Hexyl cinnamic aldehyde | 12.0 |
| Iso E Super ®³⁾ | 16.0 |
| Verdyl acetate | 24.0 |
| Lilial ®⁴⁾ | 37.0 |

1)Trademark from Firmenich; pentadecenolide, origin: Firmenich SA, Geneva, Switzerland
²⁾Trademark from Firmenich; Methyl-cis-3-oxo-2-pentyl-1-cyclopentane acetate, origin: Firmenich SA, Geneva, Switzerland
³⁾Trademark from IFF; 7-acetyl, 1,2,3,4,5,6,7,8-octahydro-1,1,6,7-tetramethyl naphthalene
⁴⁾Trademark from Givaudan; 3-(4-tert-butylpheny1)-2-methylpropanal

TABLE 2
Microcapsules compositions

| Components | A | B | C | D |
|---|---|---|---|---|
| Sodium Caseinate¹⁾ | 2.5 | 2.5 | 2.5 | 2.5 |
| CaCl₂ · 2H₂O | 0.5 | 0.5 | 0.5 | 0.5 |
| Perfume A²⁾ | 30 | 30 | 30 | 30 |
| Takenate D-1 10N³⁾ | 0.3 | 0.3 | 0.9 | 0.9 |
| Transglutaminase⁴⁾ | 1.0 | 1.0 | 1.0 | 1.0 |
| Na₂CO₃ | 0 | 3.0 | 0 | 3.0 |
| CaCl₂ | 0 | 2.9 | 0 | 2.9 |

¹⁾Ramsen Food and Dairy Products LLC
²⁾See table 1
³⁾Trimethylol propane-adduct of xylylene diisocyanate, origin: Mitsui Chemicals, Inc., Japan
⁴⁾Activa TI ® origin: Ajinomoto

### Example 2

### Preparation of Microcapsules by the Process of the Invention (Calcium Phosphate as a Mineral Layer)

Microcapsules were prepared using the same protocol as in example 1 except that the biomineralization step comprises the following steps.
1) Added 15 g of the microcapsule slurry to 135 g of NH₄OH/NH₄Cl buffer solution (pH 9) and stirred at room temperature (250 rpm, 25° C.)
2) Added 17 mL of 0.18M dibasic sodium phosphate (Na₂HPO₄) over 1 hour (283 μL/min) 3) Stirred for 1 hour
4) Simultaneously added 7.5 mL of 0.3M calcium nitrate (Ca(NO₃)₂) and 7.5 mL of 0.18M sodium phosphate over 1 hour (125 μL/min each)
5) Stirred for 1 hour
6) Simultaneously added 30 mL of 0.3M calcium nitrate (Ca(NO₃)₂) and 30 mL of 0.18M sodium phosphate over 1 hour (500 μL/min each)
7) Stirred for 1 hour
8) Repeated steps 6-7 once more

### Example 3

### Stability Performance in a Shower Gel Composition

TABLE 3
Composition of the shower gel

| Ingredients | Amount (% wt) |
|---|---|
| Deionised water | 49.35 |
| EDETA B Powder¹⁾ | 0.05 |
| Carbopol ® Aqua SF-1 | 6.00 |
| polymer²⁾ | |
| Zetesol AO 328 U³⁾ | 35.00 |
| Sodium hydroxide 20% | 1.00 |
| aqueous solution | |
| Tego ® Betain F 50⁴⁾ | 8.00 |
| Kathon CG | 0.10 |
| Citric acid 40% aqueous | 0.50 |
| solution | |

¹⁾Tetrasodium EDTA; origin: BASF
²⁾Acrylates copolymer; origin: Noveon
³⁾Sodium C12-C15 Pareth Sulfate; origin: Zschimmer & Schwarz
⁴⁾Methylchloroisothiazolinone and methylisothiazolinone; origin: Rohm & Haas

### Preparation of the Shower Gel Base

In a beaker, the deionised water is added, then the EDETA B Powder is added under stirring. The carbopol aqua SF-1 polymer and Zetesol AO 328 U are added in the reaction mixture. The pH is adjusted with sodium hydroxide solution. Tego® Betain F 50, the Kathon CG and citric acid solution are added to obtain the shower gel base (pH=6.0-6.3, Viscosity: 5000-6000 cPs, LV spindle 3, speed 12).
Capsules of the present invention were dispersed in shower gel base described in table 3 to obtain a concentration of encapsulated perfume oil at 0.20%. The samples were then aged at 37° C. for 1 week to serve as an accelerated stability assessment.

### Protocol for the Stability Assessment

1 g of sample is weighted into a 20 ml headspace vial and sealed with a septum. The sample is equilibrated for 10 minutes at 65° C. The SPME fiber is exposed to the vapor phase for 20 minutes at 65° C. The SPME fiber is desorbed into a standard GC injector (splitless) for 5 minutes at 250° C. The components were then analyzed with an Agilent GCMS (5977B MSD, 7890B GC) or equivalent. All samples were compared to a free oil reference control which corresponds to 100% leakage.

### Results

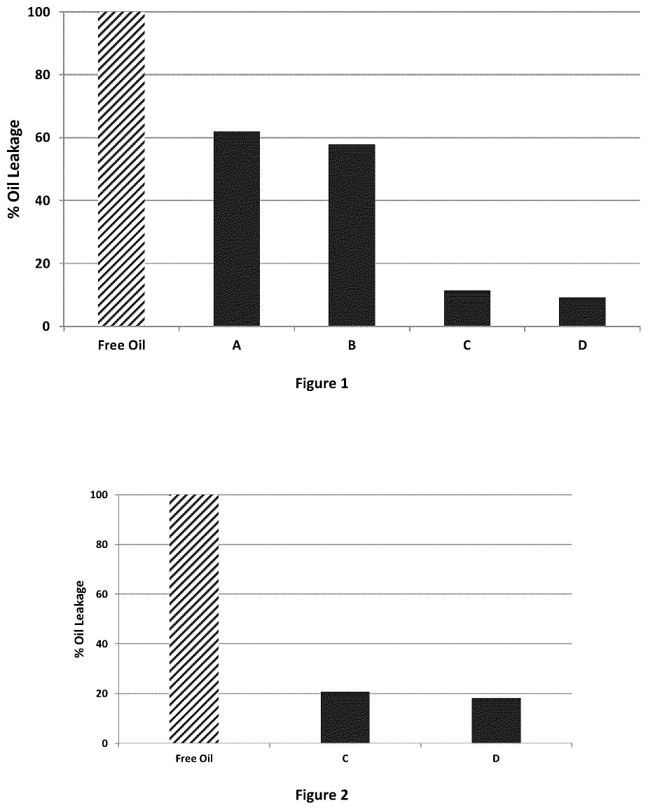

Results are shown in FIG. 1.
One can conclude from those results that even with the limited amount of polyisocyanate, the microcapsules of the invention exhibit significant encapsulation and stabilization of fragrance The capsules retain significant oil after incubation in harsh and complex application formulations for 1 week at 37° C., which serves as an accelerated stability test indicative of longer term stability and performance. Stability results are plotted against the equivalent loading of free perfume oil in shower gel.

### Example 4

### Stability Performance in a Fabric Softener Composition

Capsules of the present invention were dispersed in fabric softener base described in table 4 to obtain a concentration of encapsulated perfume oil at 0.20% and stability was evaluated after 1 week at the elevated temperature of 37° C.

TABLE 4
Fabric Softener composition

| Product | Wt % |
|---|---|
| Stepantex VL 90A | 8.88 |
| Calcium Chloride Sol. 10% | 0.36 |
| Proxel GXL | 0.04 |
| Perfume | 1 |
| Water | 89.72 |
| TOTAL | 100 |

Results are shown in FIG. 2.
One can conclude from those results that even with the limited amount of polyisocyanate, the microcapsules of the invention exhibit significant encapsulation and stabilization of fragrance The capsules retain significant oil after incubation in harsh and complex application formulations for 1 week at 37° C., which serves as an accelerated stability test indicative of longer term stability and performance. Stability results are plotted against the equivalent loading of free perfume oil in fabric softener.

### Example 5

### Olfactive Performance in a Fabric Softener Composition

On a 3″×5″ paper blotter, 0.15 g of product (fabric softener loaded with 0.2% encapsulated oil and aged for 2 weeks at 37° C.) was evenly applied onto the surface. The blotter was air dried for 24 hours before evaluation. Fragrance intensity was evaluated initially (before rubbing) and then again after rubbing the paper blotter 3 times (after rubbing).

### Evaluation Scale:

1=no odor; 2=just perceptible; 3=weak; 4=moderate; 5=strong; 6=very strong; 7=extremely strong

### Results

The intensity of the perception of the perfume on paper blotters treated with the microcapsules was evaluated by a panel of 11 trained panelists. They were asked to rate the intensity of the perfume perception on a scale ranging from 1 to 7, wherein 1 means no odour and 7 means very strong odour.

As it can be seen from FIG. 3, the microcapsules of the invention demonstrate a significant burst effect and fragrance intensity after rubbing. The low pre-rubbing intensity and high olfactive signal even after ageing the capsule slurry in the application bases for 2 weeks at the elevated temperature of 37° C. is a good indication of stability, oil retention and performance.

### Example 6

### Preparation of Microcapsules by the Process of the Invention

Similar protocol as described in Example 1 was applied to prepare microcapsules E with a composition as reported in Table 6 below. A different perfume oil (Perfume B, table 5) and different polyisocyanate concentration (0.6) was used.

TABLE 5
Perfume oil B composition

| Ingredient | Parts |
|---|---|
| Ethyl 2-methyl-pentanoate | 3.2 |
| Eucalyptol | 7.8 |
| Aldehyde C10 | 0.75 |
| 2,4-Dimethyl-3-cyclohexene- | 0.75 |
| 1-carbaldehyde ¹⁾ | |
| Citronellyl nitrile | 4.3 |
| Isobornyl acetate | 3.0 |
| Verdox ²⁾ | 9.8 |
| Citronellyl acetate | 1.3 |
| 2-methylundecanal | 3.0 |
| Diphenyloxide | 0.8 |
| Aldehyde C12 | 1.3 |
| Dicyclopentadiene acetate | 9.85 |
| Ionone beta | 3.3 |
| Undecalactone gamma | 18.75 |
| Hexyl salicylate | 15.9 |
| Benzyl salicylate | 16.2 |

¹⁾ Origin: Firmenich SA, Geneva, Switzerland
²⁾ Trademark from IFF; 2-tert-butyl-1-cyclohexyl acetate

### Example 7

### Preparation of Microcapsules by the Process of the Invention

Microcapsules F-J were prepared according to the following protocol.
1) Sodium caseinate and/or whey protein is dissolved in DI water at RT.
2) Calcium chloride (aqueous solution) is slowly added to the protein solution and stirred at RT for ˜15 min.
3) The emulsifier solution is combined with a perfume oil (see table 5) containing a polyisocyanate (Takenate® D-110N) and homogenized (10,000 rpm for 2 min).
4) The emulsion is then transferred to a reactor, pH adjusted to ˜6.5 w/NaOH, and heated to 45° C.
5) Transglutaminase (aqueous solution) is added to the reactor and it is stirred for 3 hr at 45° C.
6) The pH is adjusted to ˜5.4 w/HCl and then heated to 85° C.
7) The reactor is stirred at 85° C. for 60 min before cooling to RT.

TABLE 6
Microcapsules compositions

| | Components | | | | | |
|---|---|---|---|---|---|---|
| | E | F | G | H | I | J |
| Sodium Caseinate¹⁾ | 2.5 | 1.875 | 1.25 | 0.625 | 0 | 1.25 |
| Whey Protein²⁾ | 0 | 0.625 | 1.25 | 1.875 | 2.5 | 1.25 |
| CaCl₂•2H₂O | 0.5 | 0.5 | 0.5 | 0.5 | 0.5 | 0.5 |
| Perfume B³⁾ | 30 | 30 | 30 | 30 | 30 | 30 |
| Takenate D-110N⁴⁾ | 0.6 | 0.6 | 0.6 | 0.6 | 0.6 | 0 |
| Transglutaminase⁵⁾ | 1.0 | 1.0 | 1.0 | 1.0 | 1.0 | 1.0 |

⁵⁾Ramsen Food and Dairy Products LLC
6) Agropur Dairy Cooperative
7) See table 5
8) Trimethylol propane-adduct of xylylene diisocyanate, origin: Mitsui Chemicals, Inc., Japan
9) Activa TI ® origin: Ajinomoto

### Example 8

### Preparation of Microcapsules by the Process of the Invention (Calcium Phosphate as a Mineral Layer)

Microcapsules K-M were prepared using the same protocol as in example 7 with a biomineralization step that is the same protocol as in example 2.

TABLE 7
Microcapsules compositions

| Components | K | L | M | N | O |
|---|---|---|---|---|---|
| Sodium Caseinate¹⁾ | 1.25 | 0.625 | 1.25 | 2.5 | 0 |
| Whey Protein²⁾ | 1.25 | 1.875 | 1.25 | 0 | 2.5 |
| CaCl₂ · 2H₂O | 0.5 | 0.5 | 0.5 | 0.5 | 0.5 |
| Perfume B³⁾ | 30 | 30 | 30 | 30 | 30 |
| Takenate D-110N⁴⁾ | 0.6 | 0.6 | 0 | 0.6 | 0.6 |
| Transglutaminase⁵⁾ | 1.0 | 1.0 | 1.0 | 1.0 | 1.0 |
| Na₂HPO₄ | 17.7 | 17.7 | 17.7 | 17.7 | 17.7 |
| Ca(NO₃)₂ | 33.5 | 33.5 | 33.5 | 33.5 | 33.5 |

¹⁾Ramsen Food and Dairy Products LLC
²⁾Agropur Dairy Cooperative
³⁾See table 5
⁴⁾Trimethylol propane-adduct of xylylene diisocyanate, origin: Mitsui Chemicals, Inc., Japan
⁵⁾Activa TI ® origin: Ajinomoto

### Example 9

### Stability Performance in a Fabric Softener Composition

Capsules of the present invention were dispersed in fabric softener base described in table 4 to obtain a concentration of encapsulated perfume oil at 0.20% and stability was evaluated after 1 month at the elevated temperature of 37° C.

### Protocol for the Stability Assessment

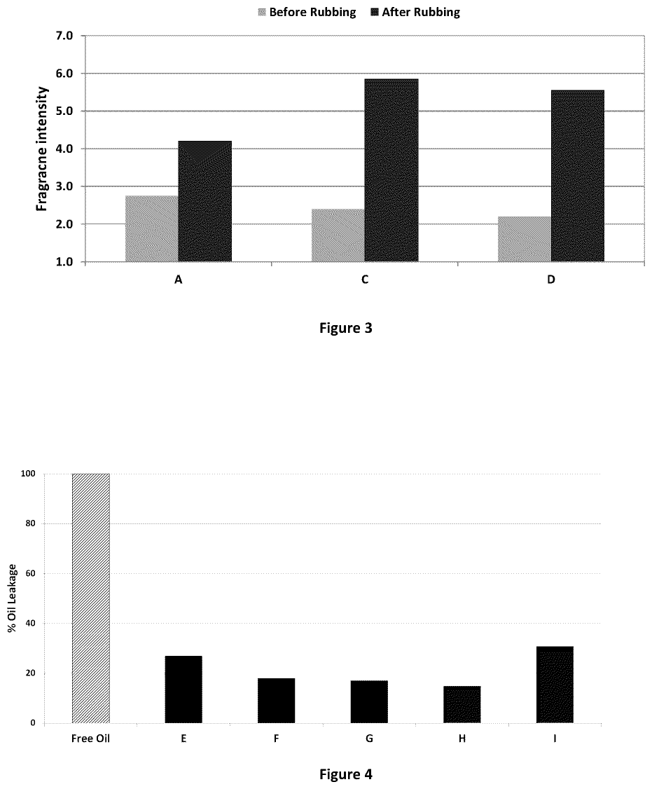

1 g of sample is weighted into a 20 mL scintillation vial. 4 mL of water are added and mixed for 5 min at 480 rpm on an IKA KS130 orbital shaker. 5 mL of extraction solvent (90% isooctane/10% ether with 150 ppm 1,4-dibromobenzene) are added and mixed for 15 min at 480 rpm on an IKA KS130 orbital shaker. Transfer to a 15 mL centrifuge tube and spin for 60 min at 6000 rcf. The supernatant with an Agilent GCMS (5977B MSD, 7890B GC) or equivalent is analyzed. All samples are compared to a free oil reference control which corresponds to 1⁰⁰% leakage.
Results are shown in FIG. 4.
One can conclude from FIG. 4, that even with the limited amount of polyisocyanate, microcapsules of the invention exhibit significant encapsulation and stabilization of fragrance oil. The capsules retain significant oil after incubation in harsh and complex application formulations for 1 month at 37° C., which serves as an accelerated stability test indicative of longer term stability and performance. Stability results are plotted against the equivalent loading of free perfume oil in fabric softener applications. Additionally, one can see from these results that microcapsule F-H, which combines sodium caseinate and whey protein show the best leakage stability.

### Example 10

### Olfactive Performance in a Fabric Softener Composition

A load of towels (24) was washed with 36 g of unperfumed detergent followed by 15 g of fabric softener loaded with 0.116% encapsulated oil (perfume B) from capsules E, F, G, or H and the towels were line-dried for 24 hours. Panelists evaluated their own set of towels and rated fragrance intensity before and after rubbing on an anchored linear labeled line scale.

### Evaluation Scale:

1=no odor; 2=just perceptible; 3=weak; 4=moderate; 5=strong; 6=very strong; 7=extremely strong

### Results

The intensity of the perception of the perfume on dried towels treated with the microcapsules was evaluated by a panel of 18 trained panelists. They were asked to rate the intensity of the perfume perception on a scale ranging from 1 to 7, wherein 1 means no odor and 7 means very strong odor.

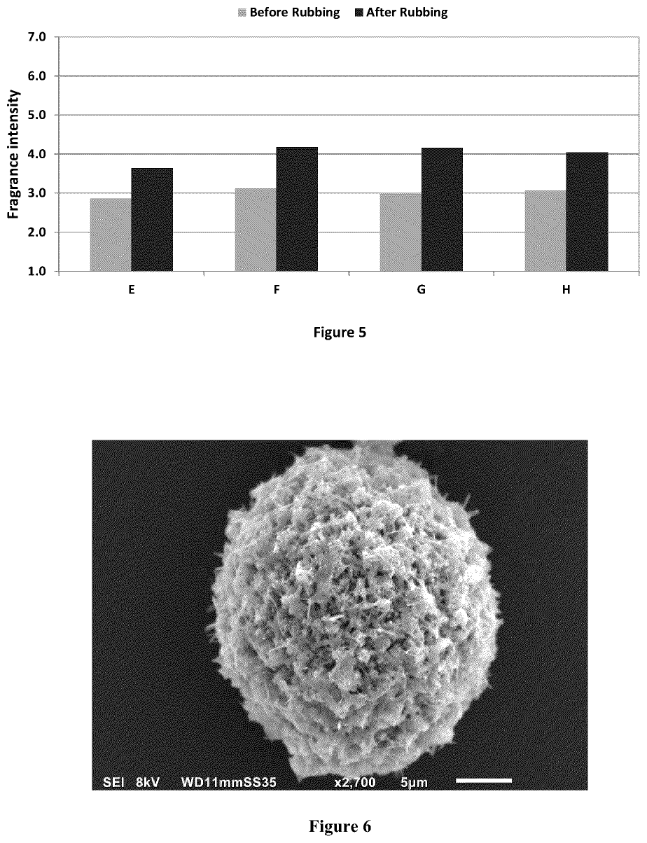

As it can be seen from FIG. 5, the microcapsules of the invention demonstrate a significant burst effect after rubbing. The low pre-rubbing intensity and high olfactive signal even after ageing the capsule slurry in the application base at the elevated temperature of 37° C. is a good indication of stability, oil retention and performance.

### Example 11

### Spray Dried Capsules

Microcapsules N were spray dried using a lab-scale Büchi B-290 Mini Spray Dyer, aspirated with compressed air at a rate set between 70% and 90% of the maximum aspiration rate, and an inlet temperature set to 200° C. Approximately 50-200 g of rinsed and condensed microcapsule slurry is pumped into the spray dryer at a pump rate set at 5-15% of the maximum pump rate. Once all slurry has been pumped into the system, the spray dryer is cooled and the dried powder collected.

### Example 12

### Capsules Characterization

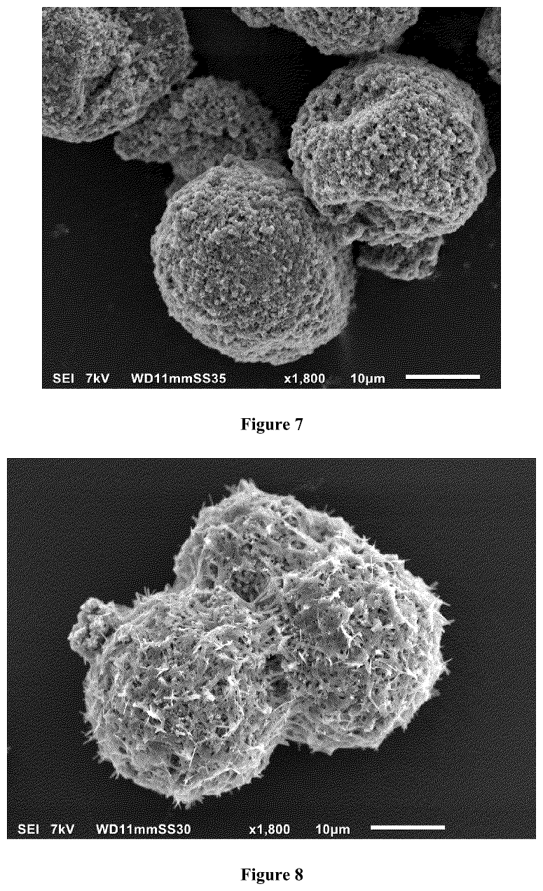

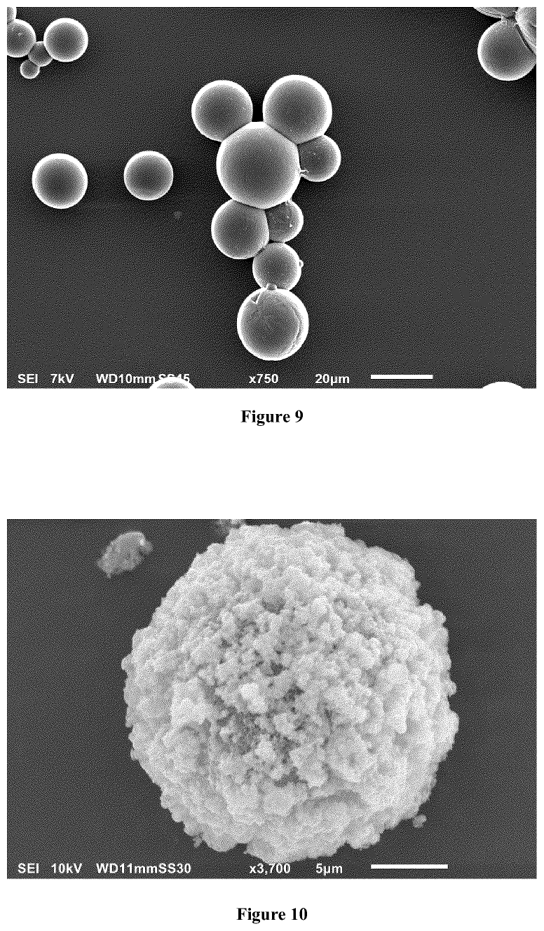

To image the microcapsules, dilute capsule slurries were dried onto carbon tape, which was adhered to aluminum stubs and then sputter coated with a gold/palladium plasma. The stubs were placed into a scanning electron microscope (JEOL 6010 PLUS LA) for analysis. Images of mineralized capsules K, N, and O are shown respectively in FIGS. 6, 7, and 8 to illustrate that stable, robust, rough mineralized microcapsules can be generated by growing a spinulose mineral coating onto smooth polyurea microcapsule scaffolds.
By contrast, capsules E in FIG. 9 have a smooth, unmodified surface.
A spray dried version of capsule N is shown in FIG. 10.
A polyisocyanate-free capsule J is shown in FIG. 11.

### Example 13

### Olfactive Performance in an Antiperspirant Roll-on Composition

Capsules are incorporated at the required dosage (corresponding to an encapsulated perfume oil at 0.20%) in the following composition.

TABLE 8
Antiperspirant roll-on composition

| Ingredients | Amount (% Wt) |
|---|---|
| Steareth-2 | 3.25 |
| Steareth-21 | 0.75 |
| PPG-15 Stearyl Ether | 4 |
| Deionized Water | 52 |
| Aluminum Chlorohydrate | 40 |
| (50% aqueous solution) | |

On a 3″×5″ paper blotter, 0.15 g of product (AP roll-on base loaded with 0.2% encapsulated oil) was evenly applied onto the surface. The blotter was air dried for 24 hours before evaluation. Fragrance intensity was evaluated initially (before rubbing) and then again after rubbing the paper blotter 3 times (after rubbing).

### Evaluation Scale:

1=no odor; 2=just perceptible; 3=weak; 4=moderate; 5=strong; 6=very strong; 7=extremely strong

### Results

The intensity of the perception of the perfume on dried blotters treated with the microcapsules was evaluated by a panel of 14 trained panelists. They were asked to rate the intensity of the perfume perception on a scale ranging from 1 to 7, wherein 1 means no odor and 7 means very strong odor.

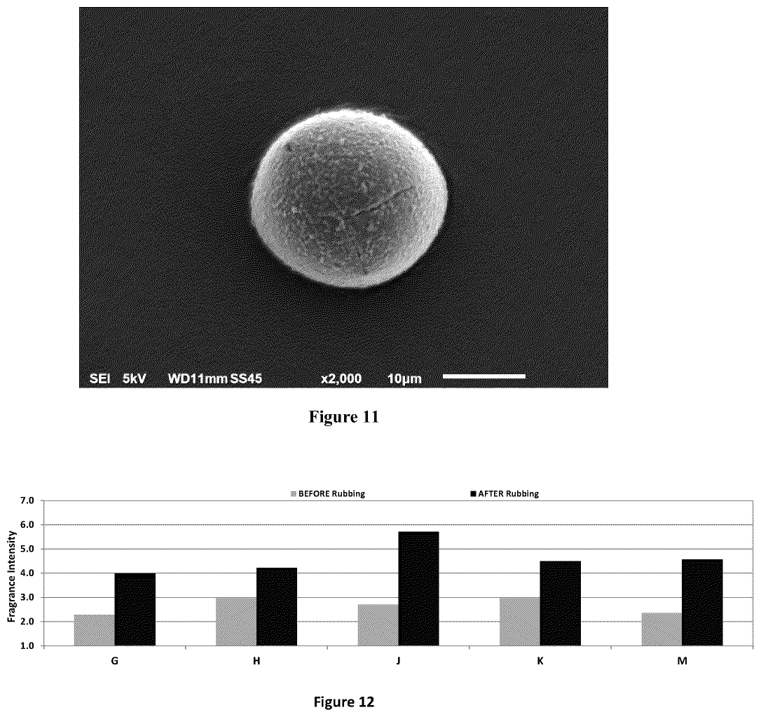

As it can be seen from FIG. 12, the microcapsules of the invention demonstrate a significant burst effect after rubbing. The low pre-rubbing intensity and high olfactive signal is a good indication of stability, oil retention and performance.

### Example 14

### Olfactive Performance in a Leave-on Hair Conditioner Composition

TABLE 9
Leave-on conditioner composition

| Ingredients | Amount (% Wt) |
|---|---|
| Water | 95.5 |
| Salcare SC 91 | 1 |
| Aculyn 46 | 1 |
| Wacker-Belsil | 0.5 |
| DMC 6038 | |
| Phenonip | 0.5 |
| Mirasil ADM-E | 1.5 |

### Hair Swatch Treatment and Sensory Evaluation Protocol (Leave-on)

Capsules are incorporated at the required dosage (corresponding to an encapsulated perfume oil at 0.20%) in the leave-on base with ample stirring at room temperature. Clean, dry, 10 g hair swatches are wetted with 37° C. warm tap water for 30 seconds. 2.5 g of unperfumed shampoo is applied per hair swatch and lathered for 30 seconds before rinsing for 30 seconds (15 seconds per side of the swatch) under warm running water directed at the top of the hair swatch mount (flow rate=4 L/min). The excess water is gently squeezed out. 1 g of leave-on product is then applied per hair swatch, and is gently rubbed and distributed into the hair swatch evenly with gloved hands for 1 min. The hair swatch is then combed before being placed on a drying rack to air dry. The hair swatches are evaluated after 24 hours by expert panelists using an intensity scale of 1-7 as follows: 1) Imperceptible; 2) Slightly Perceptible; 3) Weak; 4) Medium; 5) Sustained; 6) Intense; 7) Very Intense.

### Evaluation Scale:

1=no odor; 2=just perceptible; 3=weak; 4=moderate; 5=strong; 6=very strong; 7=extremely strong.

### Results

The intensity of the perception of the perfume on dried towels treated with the microcapsules was evaluated by a panel of 15 trained panelists. They were asked to rate the intensity of the perfume perception on a scale ranging from 1 to 7, wherein 1 means no odor and 7 means very strong odor.

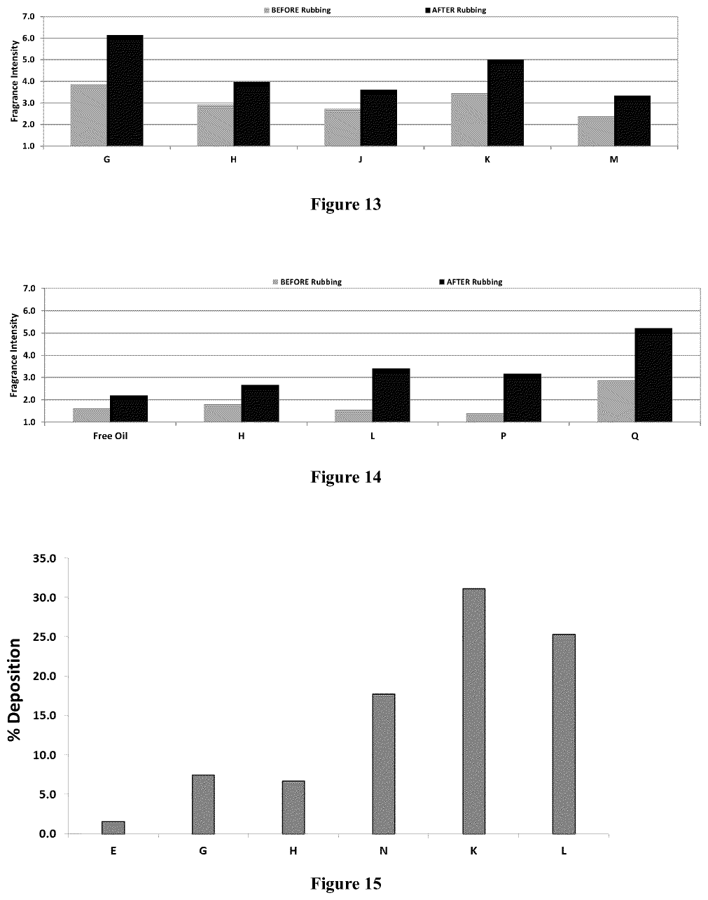

As it can be seen from FIG. 13 the microcapsules of the invention demonstrate a significant burst effect after rubbing. The low pre-rubbing intensity and high olfactive signal is a good indication of stability, oil retention and performance.

### Example 15

### Addition of a Cationic Coating to Capsules of the Invention

Process for preparing microcapsules P and Q correspond respectively to the process for preparing microcapsules H and L except that an additional step of adding a cationic copolymer, namely acrylamidopropyltrimonium chloride/acrylamide copolymer (Salcare® SC60, origin BASF) (3 wt/% in water) has been carried out at the end of the process.

TABLE 10
Microcapsules composition

| Components | P | Q |
|---|---|---|
| Sodium Caseinate¹⁾ | 0.625 | 0.625 |
| Whey Protein²⁾ | 1.875 | 1.875 |
| CaCl₂ · 2H₂O | 0.5 | 0.5 |
| Perfume B³⁾ | 30 | 30 |
| Takenate D-110N⁴⁾ | 0.6 | 0.6 |
| Transglutaminase⁵⁾ | 1.25 | 0.625 |
| Na₂HPO₄ | 0 | 17.7 |
| Ca(NO₃)₂ | 0 | 33.5 |
| Salcare ^{®} SC60⁶⁾ | 1.5 | 1.5 |

¹⁾Ramsen Food and Dairy Products LLC
²⁾Agropur Dairy Cooperative
³⁾See table 5
⁴⁾Trimethylol propane-adduct of xylylene diisocyanate, origin: Mitsui Chemicals, Inc., Japan
⁵⁾Activa TI ® origin: Ajinomoto
⁶⁾acrylamidopropyltrimonium chloride/acrylamide copolymer; origin BASF

### Example 16

### Olfactive Performance in a Rinse-Off Shampoo Composition

TABLE 11
Rinse-off shampoo formulation

| Ingredients | Amount (% Wt) |
|---|---|
| Deionized Water | 45.97 |
| EDETA B POWDER | 0.05 |
| JAGUAR C14 S | 0.05 |
| UCARE POLYMER JR-400 | 0.075 |
| NaOH (10% aqueous solution) | 0.30 |
| SULFETAL LA B-E | 34.00 |
| ZETESOL LA | 9.25 |
| TEGOBETAINE F-50 | 2.00 |
| XIAMETER MEM-1691 | 2.50 |
| CETYL ALCOHOL | 1.20 |
| COMPERLAN100 | 1.50 |
| CUTINA AGS | 2.00 |
| KATHON CG | 0.10 |
| PANTHENOL 75% | 0.10 |
| WATER DEIONISED | 0.30 |
| SODIUM CHLORIDE 25% | 0.60 |
| (aqueous solution) | |

### Hair Swatch Treatment and Sensory Evaluation Protocol (Rinse-Off)

Capsules are incorporated at the required dosage (corresponding to an encapsulated perfume oil at 0.5%) in the rinse-off base with sample stirring at room temperature. Clean, dry, 10 g hair swatches are wetted with 37° C. warm tap water for 30 seconds. 1 g of rinse-off product is applied per hair swatch, and is gently rubbed and distributed into the hair swatch evenly with gloved hands. To rinse the hair swatches, the hair swatches are double-rinsed using a sequential beaker wash involving dipping and fanning of the hair swatch in clean warm water three times per movement, followed by a 30 second rinse (15 seconds per side of the swatch) under warm running water directed at the top of the hair swatch mount (flow rate=4 L/min). The hair swatches are not squeezed dry. The sample application, distribution and rinsing are repeated a second time before placing the hair swatches on a drying rack to air dry. The hair swatches are evaluated after 24 hours by expert panelists using an intensity scale of 1-7 as follows: 1) Imperceptible; 2) Slightly Perceptible; 3) Weak; 4) Medium; 5) Sustained; 6) Intense; 7) Very Intense.

### Evaluation Scale:

1=no odor; 2=just perceptible; 3=weak; 4=moderate; 5=strong; 6=very strong; 7=Extremely Strong

### Results

The intensity of the perception of the perfume on dried towels treated with the microcapsules was evaluated by a panel of 16 trained panelists. They were asked to rate the intensity of the perfume perception on a scale ranging from 1 to 7, wherein 1 means no odor and 7 means very strong odor.

As it can be seen from FIG. 14, the microcapsules of the invention demonstrate a significant burst effect after rubbing. The low pre-rubbing intensity and high olfactive signal is a good indication of stability, oil retention and performance.

### Example 17

### Deposition Testing on Hair

For the quantification of deposition, the following procedure was used. A 500 mg mini hair swatch was wet with 40 mL of tap water (37-39° C.) aimed at the mount with a 140 mL syringe. The excess water was gently squeezed out once and 0.1 mL of a model surfactant mixture containing microcapsules loaded with a UV tracer (Uvinul A Plus) was applied with a 100 μL positive displacement pipet. The surfactant mixture was distributed with 10 horizontal and 10 vertical passes. The swatch was then rinsed with 100 mL of tap water (37-39° C.) with 50 mL applied to each side of the swatch aimed at the mount. The excess water was gently squeezed out and the hair swatch was then cut into a pre-weighed 20 mL scintillation vial. This process was repeated 2 more times and then the vials containing the cut hair were dried in a vacuum oven @50-60° C. (100 Torr) for at least 5 hours. After the drying process, the vials were again weighed to determine the mass of the hair in the vials. Controls were also prepared by adding 0.1 mL of the model surfactant mixture containing capsules to an empty vial. 4 mL of 200-proof ethanol were then added to each vial and they were subjected to 60 minutes of sonication. After sonication, the samples were filtered through a 0.45 μm PTFE filter and analyzed with a HPLC using a UV detector. To determine the percent deposition of microcapsules from a model surfactant mixture, the amount of Uvinul extracted from the hair samples was compared to the amount of Uvinul extracted from the control samples.

TABLE 12
Model surfactant mixture composition

| Ingredients | Active Amount (% wt) | Function |
|---|---|---|
| Sodium Laurel Ether | 12 | Anionic |
| Sulfate (SLES) | | Surfactant |
| Cocamidopropyl | 3 | Amphoteric |
| Betaine (CAPB) | | Surfactant |
| Salcare ® SC | 0.5 | Deposition |
| 60 1) Polymer¹⁾ | | Aid |
| Water | 84.5 | Solvent |

¹⁾acrylamidopropyltrimonium chloride/acrylamide copolymer; origin BASF

### Results

Deposition onto hair swatches was measured from this simplified model surfactant mixture which is meant to be representative of personal cleansing formulations such as shampoo or shower gel. Results are shown in FIG. 15.
The data illustrated in FIG. 15 demonstrate that the capsules according to the invention (Capsules E, G, and H) deposit a quantifiable amount of fragrance oil onto hair swatches from a model surfactant mixture and that the addition of a mineral layer to these capsules, according to the invention (Capsules N, K, and L, respectively) increases the deposition of oil onto the hair swatches by up to five times.

### Example 18

### Stability of Mineral Coating in Hydrogen Peroxide for Oral Care Applications

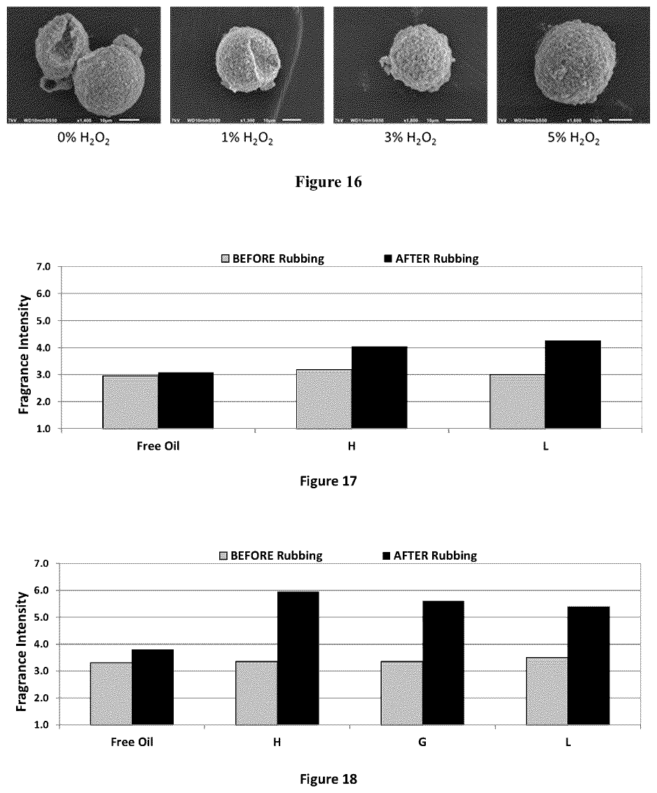

Stability protocol is as follows: 100 mg of microcapsule slurry was introduced into 10 ml of a solution of hydrogen peroxide pH adjusted to 6.5 and gently stirred before incubating samples for one month at 22° C. Microcapsules were then observed using scanning electron microscopy to determine if any physical deterioration of the mineral shell was observable. FIG. 16 demonstrates that the outer mineral coating is not affected by the pH or hydrogen peroxide content found in many oral care applications.

### Example 19

### Rinse-Off Conditioner Composition

Fragranced microcapsules H were added to the rinse-off composition above. 10 g Caucasian brown hair swatches were used with a length of 20 cm and fixed with a flat metal clip. Caucasian hair, flat bundled, was chosen for this evaluation because Caucasian hair is rather thin in diameter and the application of viscous conditioner compositions can be guaranteed to be more reproducible compared to thick and course Asian hair. The hair swatches were rinsed with warm tap water (37° C.) and excess water was squeezed off manually. 1 g of the rinse-off product was applied on the swatch and distributed manually during 30 seconds, wearing nitrile gloves. Swatches were then air dried on a drying rack during 24 hours. Olfactive evaluation was carried out by a group of 8 panelists on the dried swatches before and after combing. The intensity was reported on a scale from 1-7 (1=no odor, 7=maximum odor intensity). The average of 8 panelist evaluations is reported.

TABLE 13
rinse-off conditioner composition

| | | Concentration |
|---|---|---|
| | Ingredients | [wt %] |
| A | Water deionized | 81.8 |
| | Behentrimonium Chloride¹⁾ | 2.5 |
| | Hydroxyethylcellulose²⁾ | 1.5 |
| B | Cetearyl Alcohol³⁾ | 4 |
| | Glyceryl Stearate (and) PEG-100 Stearate⁴⁾ | 2 |
| | Behentrimonium Methosulfate (and) | 4 |
| | Cetyl alcohol (and) | |
| | Butylene Glycol⁵⁾ | |
| | Ethoxy (20) Stearyl Alcohol⁶⁾ | 1 |
| C | Amodimethicone (and) Trideceth-12 (and) | 3 |
| | Cetrimonium Chloride⁷⁾ | |
| | Chlorhexidine Digluconate⁸⁾ | 0.2 |
| | 20% aqueous solution | |
| D | Citric acid 10% aqueous sol. till pH 3.5-4 | q. s. |
| | TOTAL: | 100 |

¹⁾Genamin KDMP, Clariant
²⁾Tylose H10 Y G4, Shin Etsu
³⁾Lanette O, BASF
⁴⁾Arlacel 165, Croda
⁵⁾Incroquat Behenyl TMS-50-PA-(MH), Croda
⁶⁾Brij S20, Croda
⁷⁾Xiameter MEM-949, Dow Corning
8)Alfa Aesar

Ingredients of Phase A are mixed until a uniform mixture was obtained. Tylose is allowed to completely dissolve. Then the mixture is heated up to 70-75° C. Ingredients of Phase B are combined and melted at 70-75° C. Then ingredients of Phase B are added to Phase A with good agitation and the mixing is continued until cooled down to 60° C. Then, ingredients of Phase C are added while agitating and keeping mixing until the mixture cooled down to 40° C. The pH is adjusted with citric acid solution till pH: 3.5-4.0.

TABLE 14
Olfactive performance in a rinse-off composition

| | Before Combing | after combing |
|---|---|---|
| 0.7% Capsules H | 3 | 6 |

One can note from Table 14 that microcapsules according to the invention show a rubbing effect.

### Example 20

### Liquid Detergent Composition

A sufficient amount of microcapsules H (0.19 g) was weighed and mixed in a 35 g dose of liquid detergent (Table 15) to add the equivalent of 0.15% perfume.

TABLE 15
Liquid detergent composition

| | Concentration |
|---|---|
| Ingredients | [wt %] |
| Sodium C₁₄₋₁₇ Alkyl Sec Sulfonate¹⁾ | 7 |
| Fatty acids, C₁₂₋₁₈ and C₁₈-unsaturated²⁾ | 7.5 |
| C_{12/14} fatty alcohol polyglycol | 17 |
| ether with 7 mol EO³⁾ | |
| Triethanolamine | 7.5 |
| Propylene Glycol | 11 |
| Citric acid | 6.5 |
| Potassium Hydroxyde | 9.5 |
| Properase L⁴⁾ | 0.2 |
| Puradax EG L⁴⁾ | 0.2 |
| Purastar ST L⁴⁾ | 0.2 |
| Acrylates/Steareth-20 Methacrylate | 6 |
| structuring Crosspo1ymer⁵⁾ | |
| Deionized Water | 27.4 |

¹⁾Hostapur SAS 60; Origin: Clariant
²⁾Edenor K 12-18; Origin: Cognis
³⁾Genapol LA 070; Origin: Clariant
⁴⁾Origin: Genencor International
⁵⁾Aculyn 88; Origin: Dow Chemical

### Protocol

Fabrics (2.0 kg of cotton terry towels) were washed at 40° C. in a standard European horizontal axis machine (Miele Novotronic W 900-79 CH) with a 35 g dose of liquid detergent containing 0.53% microcapsules slurry. After the wash, fabrics were line-dried overnight before the odor intensity of the cotton towels was evaluated by a panel of 8 trained panelists. The panelists were asked to rate the odor intensity of the towels before & after gentle rubbing of the fabrics by hand on a scale from 1 to 7, 1 corresponding to odorless and 7 corresponding to a very strong odor.

### Results

TABLE 16
Olfactive intensity on towels (before and after rubbing)

| | Line Drying | |
|---|---|---|
| Overall Perfume Intensity | 1 day Before | 1 day After |
| Fresh sample | Rubbing | Rubbing |
| 35 g liquid detergent with 0.53% | 2.5 | 3.5 |
| microcapsule slurry H | | |

### Example 21

### Olfactive Performance in High Ethanol EdT Composition

TABLE 17
High Ethanol EdT composition

| Ingredients | Amount (% Wt) |
|---|---|
| Ethanol 40B | 78.00 |
| Capsule Slurry | equivalent of 1% perfume oil |
| Deionized Water | remaining quantity based on the total |
| | weight of capsule slurry |

On a 3″×5″ paper blotter, 160 uL (˜0.2 g) of product (High Ethanol EdT base loaded with 1% encapsulated perfume oil) was evenly applied onto the surface. The blotter was air dried for 1 hour and then for 4 hours on a precision hot plate pre-heated to 32° C., totaling to 5 hours drying time before evaluation. Fragrance intensity was evaluated initially (before rubbing) and then again after rubbing the paper blotter 3 times (after rubbing).

### Evaluation Scale:

1=no odor; 2=just perceptible; 3=weak; 4=moderate; 5=strong; 6=very strong; 7=Extremely Strong

### Results

The intensity of the perception of the perfume on dried blotters treated with microcapsules H, and L was evaluated by a panel of 20 trained panelists. They were asked to rate the intensity of the perfume perception on a scale ranging from 1 to 7, wherein 1 means no odor and 7 means very strong odor.

As it can be seen from FIG. 17, the microcapsules of the invention demonstrate a significant burst effect after rubbing in comparison to the intensity delta of the free oil control.

### Example 22

### Olfactive Performance in Low Ethanol EdT Composition

TABLE 18
Low Ethanol EdT composition

| Ingredients | Amount (% Wt) |
|---|---|
| Ethanol 40B | 40.00 |
| Capsule Slurry | equivalent of 1% perfume oil |
| Deionized Water | remaining quantity based on the total |
| | weight of capsule slurry |

On a 3″×5″ paper blotter, 160 uL (˜0.2 g) of product (Low Ethanol EdT base loaded with 1% encapsulated perfume oil) was evenly applied onto the surface. The blotter was air dried for 1 hour and then for 4 hours on a precision hot plate pre-heated to 32° C., totaling to 5 hours drying time before evaluation. Fragrance intensity was evaluated initially (before rubbing) and then again after rubbing the paper blotter 3 times (after rubbing).

### Evaluation Scale:

1=no odor; 2=just perceptible; 3=weak; 4=moderate; 5=strong; 6=very strong; 7=Extremely Strong

### Results

The intensity of the perception of the perfume on dried blotters treated with microcapsules H, G, and L was evaluated by a panel of 20 trained panelists. They were asked to rate the intensity of the perfume perception on a scale ranging from 1 to 7, wherein 1 means no odor and 7 means very strong odor.

As it can be seen from FIG. 18, the microcapsules of the invention demonstrate a significant burst effect after rubbing in comparison to the intensity delta of the free oil control.

### Example 23

### Spray-Dried Microcapsules Preparation

Emulsions 1-5 having the following ingredients are prepared.

TABLE 19
Composition of Emulsions 1-5 and composition
of granulated powder 1-5 after spray-drying

| | Emulsion | Emulsion | Emulsion | Emulsion | Emulsion |
|---|---|---|---|---|---|
| Ingredients | 1 | 2 | 3 | 4 | 5 |
| Modified | 2.6% | 2.6% | 2.6% | 12.5% | 2% |
| starch ¹⁾ | | | | | |
| Maltodextrin ²⁾ | 26.8% | 22.8% | 19.3% | 0% | 19.1% |
| Maltose ³⁾ | 0% | 0% | 0% | 7.9% | 0% |
| Citric Acid | 0% | 0% | 0% | 1% | 0% |
| Tripotassium | 0% | 0% | 0% | 1.9% | 0% |
| Citrate | | | | | |
| Microcapsules | 12.0% | 24% | 37.0% | 8.9% | 56.2% |
| E-K | | | | | |
| Silica ⁴⁾ | 1.1% | 1.1% | 1.1% | 0% | 0% |
| Free Perfume | 0% | 0% | 0% | 11% | 0% |
| C ⁵⁾ | | | | | |
| Water | 57.6% | 49.6% | 40.1% | 56.9% | 22.7% |
| | Granule | Granule | Granule | Granule | Granule |
| | 1 | 2 | 3 | 4 | 5 |
| Modified | 7.5% | 7.4% | 7.2% | 31.6% | 4.9% |
| starch ¹⁾ | | | | | |
| Maltodextrin ²⁾ | 77.4% | 65.5% | 53.8% | 0% | 44.7% |
| Maltose | 0% | 0% | 0% | 20.9% | |
| Citric Acid | 0% | 0% | 0% | 2.6% | 0% |
| Tripotassium | 0% | 0% | 0% | 4.9% | 0% |
| citrate | | | | | |
| Encapsulated | 0% | 0% | 0% | 28.1% | 0% |
| perfume C | | | | | |
| Microcapsules | 12.% | 24.1% | 36.1% | 9.8% | 48.4% |
| E-K | | | | | |
| Silica | 3.0 | 3.0% | 2.9% | 2.0% | 2% |
| Fragrance | 10.1% | 20.1% | 30% | 35.8% | 40.2% |
| loading in | | | | | |
| powder after | | | | | |
| spray-drying | | | | | |

¹⁾ CapsulTM, Ingredion
²⁾ Maltodextrin 10DE origin: Roquette
³⁾ Maltose, Lehmann & Voss
⁴⁾ Silica, Evonik
⁵⁾ see table 20

TABLE 20
Composition of Perfume C

| Component | % |
|---|---|
| ACÉTATE DE 4-(1,1-DIMÉTHYLÉTHYL)- | 14.50 |
| 1-CYCLOHEXYLE ¹⁾ | |
| LINALOL BJ | 10.50 |
| LILIAL ®²⁾ | 10.00 |
| ISO E SUPER ³⁾ | 10.00 |
| CITRONELLYL NITRILE | 9.00 |
| DIPHENYLOXYDE | 6.50 |
| ISOBORNYL ACETATE | 6.00 |
| BETA IONONE | 6.00 |
| TRICYCLO[5.2.1.0~2,6~]DEC-3-EN- 8-YL | 5.50 |
| ACETATE (A) + | |
| TRICYCLO[5.2.1.0~2,6~]DEC-4-EN-8-YL | |
| ACETATE (B) ⁴⁾ | |
| ETHER MT | 4.00 |
| HEDIONEO ® ⁵⁾ | 4.00 |
| GERANIOL 60 | 3.00 |
| CITRAL | 2.50 |
| ALDEHYDE C 10 | 2.50 |
| ALLYL HEPTANOATE | 2.50 |
| ETHYL METHYL-2-BUTYRATE | 1.50 |
| GERANYL ACETATE | 1.00 |
| 2,4-DIMETHYL-3-CYCLOHEXENE-1- | 1.00 |
| CARBALDEHYDE ⁶⁾ | |

¹⁾ Firmenich SA, Switzerland
²⁾3-(4-tert-butylphenyl)-2-methylpropanal, Givaudan SA, Vernier, Switzerland
³⁾ 1-(octahydro-2,3,8,8-tetramethyl-2-naphtalenyl)-1-ethanone, International Flavors & Fragrances, USA
⁴⁾ Firmenich SA, Switzerland
⁵⁾ Methyl dihydrojasmonate, Firmenich SA, Switzerland
⁶⁾ Firmenich SA, Switzerland

Components for the polymeric matrix (Maltodextrin and Capsul™, or Capsul™, citric acid and tripotassium citrate) are added in water at 45-50° C. until complete dissolution.
For emulsion 4, free perfume C is added to the aqueous phase.
Microcapsules slurry is added to the obtained mixture. Then, the resulting mixture is then mixed gently at 25° C. (room temperature).
Granulated powder A-E are prepared by spray-drying Emulsion A-E using a Sodeva Spray Dryer (Origin France), with an air inlet temperature set to 215° C. and a throughput set to 500 ml per hour. The air outlet temperature is of 105° C. The emulsion before atomization is at ambient temperature.

### Example 24

### Liquid Scent Booster Composition

A sufficient amount of microcapsule slurry E, F, G, H, I, J or K is weighed and mixed in a liquid scent booster (Table 21) to add the equivalent of 0.2% perfume.

TABLE 21
Liquid scent booster composition

| | Amount (% wt) | | | | | |
|---|---|---|---|---|---|---|
| Ingredients | 1 | 2 | 3 | 4 | 5 | 6 |
| Water | 71.20% | 89.5% | 78.8% | 79.4% | 70% | 70% |
| Propylene | 20.30% | — | — | — | 20% | 20% |
| glycol | | | | | | |
| Polyethylene | 4.00% | 6% | | | | |
| glycol ethers | | | | | | |
| of decyl | | | | | | |
| alcohol¹⁾ | | | | | | |
| Polyethylene | | | | | 4.00% | 4.00% |
| glycol ether | | | | | | |
| of Lauryl | | | | | | |
| Alcohol²⁾ | | | | | | |
| alkyl | | | 8.30% | 7.7% | | |
| polyglucoside | | | | | | |
| C8-C10³⁾ | | | | | | |
| Deceth-3¹⁾ | 1.50% | | | | | |
| Lauryl lactate | | | | 1% | | |
| Lauric acid | | 1.5% | 1.60% | | | |
| Glyceryl | | | | | 3.00% | 3.00% |
| Caprylate | | | | | | |
| Fragrance | 3.00% | 3.0% | 3.00% | 3.00% | 3.00% | 0% |

¹⁾Deceth-8; trademark and origin: KLK Oleo
²⁾Laureth-9;; trademark and origin
³⁾Plantacare 2000UP; trademark and origin: BASF

Different ringing gel compositions are prepared (compositions 1-6) according to the following protocol.

In a first step, the aqueous phase (water), the solvent (propylene glycol) if present and surfactants are mixed together at room temperature under agitation with magnetic stirrer at 300 rpm for 5 min.

In a second step, the linker is dissolved in the hydrophobic active ingredient (fragrance) at room temperature under agitation with magnetic stirrer at 300 rpm. The resulting mixture is mixed for 5 min.

Then, the aqueous phase and the oil phase are mixed together at room temperature for 5 min leading to the formation of a transparent or opalescent ringing gel.

### Example 25

### Powder Detergent Composition

A sufficient amount of granules 1-5 is weighed and mixed in a powder detergent composition (Table 22) to add the equivalent of 0.2% perfume.

TABLE 22
Powder detergent composition

| Ingredients | Part |
|---|---|
| Anionic (Linear Alkyl Benzene | 20% |
| Sulphonates) | |
| Nonionics (Alcohol Ethoxylates (5-9 | 6% |
| ethylene oxide) | |
| Builders (zeolites, sodium carbonate) | 25% |
| Silicates | 6% |
| Sodium Sulphate | 35% |
| Others (Enzymes, Polymers, Bleach) | 7.5% |
| Spray-dried granule powder 1-5 | 0.5% |

### Example 26

### Concentrated all Purpose Cleaner Composition

A sufficient amount of microcapsule slurry E, F, G, H, I, J or K is weighed and mixed in a concentrated all-purpose cleaner composition (Table 23) to add the equivalent of 0.2% perfume.

TABLE 23
concentrated all-purpose cleaner composition

| | Amount | |
|---|---|---|
| Ingredients | (% wt) | Function |
| Ethoxylated Alcohol (C9-C11, | 20 | Non-ionic |
| 8EO) ⁽¹⁾ | | surfactant |
| Sodium Dodecyl Benzene | 16 | Anionic |
| Sulfonate ⁽²⁾ | | surfactant |
| Sodium Cumene Sulfonate ⁽³⁾ | 8 | Hydrotrope |
| Methyl chloro isothiazolinone | 0.8% | preservative |
| Methyl isothiazolinone 3.3:1 ⁽⁴⁾ | | |
| Water | 55.9 | solvent |

⁽¹⁾ Neodol 91-8 ®; trademark and origin: Shell Chemical
⁽²⁾ Biosoft D-400 ®; trademark and origin: Stepan Company
⁽³⁾ Stepanate SCS ®; trademark and origin: Stepan Company
⁽⁴⁾ Kathon CG ®; trademark and origin: Dow Chemical Company

### Example 27

### Solid Scent Booster Composition

The following compositions are prepared.

TABLE 24
Salt-based solid scent booster compositions

| Ingredients | Part |
|---|---|
| Sodium chloride | 95 |
| Spray-dried granule powder 1-5 | 5 |

TABLE 25
Urea-based solid scent booster compositions

| Ingredients | Part |
|---|---|
| Urea (beads) | 94 |
| Spray-dried granule powder 1-5 | 8 |
| Bentonite | 3 |
| Perfume | 3 |

### Example 28

### Shampoo Composition

A sufficient amount of microcapsule slurry E, F, G, H, I, J or K is weighed and mixed in a shampoo composition (Table 26) to add the equivalent of 0.2% perfume.

TABLE 26
Shampoo composition

| | | Concentration |
|---|---|---|
| | Ingredients | [wt %] |
| A | Water deionized | 44.4 |
| | Polyquaternium-10 ¹⁾ | 0.3 |
| | Glycerin 85% ²⁾ | 1 |
| | DMDM Hydantoin ³⁾ | 0.2 |
| B | Sodium Laureth Sulfate ⁴⁾ | 28 |
| | Cocamidopropyl Betaine ⁵⁾ | 3.2 |
| | Disodium Cocoamphodiacetate ⁶⁾ | 4 |
| | Ethoxy (20) Stearyl Alcohol ⁶⁾ | 1 |
| C | Sodium Laureth Sulfate ⁴⁾ | 3 |
| | Glyceryl Laureate ⁷⁾ | 0.2 |
| D | Water deionized | 1 |
| | Sodium Methylparaben ⁸⁾ | 0.1 |
| E | Sodium Chloride 10% aqueous sol. | 15 |
| | Citric acid 10% aqueous sol. | q.s. |
| | till pH 5.5-6 | |
| | Perfume | 0.5 |
| | TOTAL | 100 |

¹⁾ Ucare Polymer JR-400, Noveon
²⁾ Schweizerhall
³⁾ Glydant, Lonza
⁴⁾ Texapon NSO IS, Cognis
⁵⁾ Tego Betain F 50, Evonik
⁶⁾ Amphotensid GB 2009, Zschimmer & Schwarz
⁷⁾ Monomuls 90 L-12, Gruenau
⁸⁾ Nipagin Monosodium, NIPA

Polyquaternium-10 is dispersed in water. The remaining ingredients of phase A are mixed separately by addition of one after the other while mixing well after each adjunction. Then this pre-mix is added to the Polyquaternium-10 dispersion and was mixed for 5 min. Then Phase B and the premixed Phase C (heat to melt Monomuls 90L-12 in Texapon NSO IS) are added. The mixture is mixed well. Then, Phase D and Phase E are added while agitating. The pH was adjusted with citric acid solution till pH: 5.5-6.0.

### Example 29

### Shampoo Composition

A sufficient amount of microcapsule slurry E, F, G, H, I, J or K is weighed and mixed in a shampoo composition (Table 27) to add the equivalent of 0.2% perfume.

TABLE 27
Shampoo composition

| | | Concentration |
|---|---|---|
| | Ingredients | [wt %] |
| A | Water deionized | 45.97 |
| | Tetrasodium EDTA ¹⁾ | 0.05 |
| | Guar Hydroxypropyltrimonium Chloride ²⁾ | 0.05 |
| | Polyquaternium-10 ³⁾ | 0.075 |
| B | NaOH 10% aqueous sol. | 0.3 |
| C | Ammonium Lauryl Sulfate ⁴⁾ | 34 |
| | Ammonium Laureth Sulfate ⁵⁾ | 9.25 |
| | Cocamidopropyl Betaine ⁶⁾ | 2 |
| | Dimethicone (&) C12-13 Pareth-4 (&) | 2.5 |
| | C12-13 Pareth-23 (&) Salicylic Acid ⁷⁾ | |
| D | Cetyl Alcohol ⁸⁾ | 1.2 |
| | Cocamide MEA ⁹⁾ | 1.5 |
| | Glycol Distearate ¹⁰⁾ | 2 |
| E | Methylchloroisothiazolinone & | 0.1 |
| | Methylisothiazolinone ¹¹⁾ | |
| | D-Panthenol 75% ¹²⁾ | 0.1 |
| | Water deionized | 0.3 |
| F | Sodium Chloride 25% aqueous sol. | 0.6 |
| | TOTAL: | 100 |

¹⁾ EDETA B Powder, BASF
²⁾ Jaguar C14 S, Rhodia
³⁾ Ucare Polymer JR-400, Noveon
⁴⁾ Sulfetal LA B-E, Zschimmer & Schwarz
⁵⁾ Zetesol LA, Zschimmer & Schwarz
6) Tego Betain F 50, Evonik
⁷⁾ Xiameter MEM-1691, Dow Corning
⁸⁾ Lanette 16, BASF
⁹⁾ Comperlan 100, Cognis
¹⁰⁾ Cutina AGS, Cognis
¹¹⁾ Kathon CG, Rohm & Haas
¹²⁾ D-Panthenol, Roche

A premix comprising Guar Hydroxypropyltrimonium Chloride and Polyquaternium-10 are added to water and Tetrasodium EDTA while mixing. When the mixture is homogeneous, NaOH is added. Then, Phase C ingredients are added and the mixture was heat to 75° C. Phase D ingredients are added and mixed till homogeneous. The heating is stopped and temperature of the mixture is decreased to RT. At 45° C., ingredients of Phase E while mixing final viscosity is adjusted with 25% NaCl solution and pH of 5.5-6 is adjusted with 10% NaOH solution.

### Example 30

### Antiperspirant Spray Anhydrous Composition

A sufficient amount of microcapsule slurry E, F, G, H, I, J or K is weighed and mixed in an antiperspirant spray anhydrous composition (Table 28) to add the equivalent of 0.2% perfume.

TABLE 28
antiperspirant spray anhydrous composition

| | Amount |
|---|---|
| Ingredient | (wt %) |
| Cyclomethicone¹⁾ | 53.51 |
| Isopropyl miristate | 9.04 |
| Silica²⁾ | 1.03 |
| Quaternium-18-Hectorite³⁾ | 3.36 |
| Aluminium Chlorohydrate⁴⁾ | 33.06 |

¹⁾Dow Corning ® 345 Fluid; trademark and origin: Dow Corning
²⁾Aerosil ® 200; trademark and origin: Evonik
³⁾Bentone ® 38; trademark and origin: Elementis Specialities
⁴⁾Micro Dry Ultrafine; origin: Reheis

Using a high speed stirrer, Silica and Quaternium-18-Hectorite are added to the Isopropyl miristate and Cyclomethicone mixture. Once completely swollen, Aluminium Chlorohydrate is added portion wise under stirring until the mixture was homogeneous and without lumps. The aerosol cans are filled with 25% Suspension of the suspension and 75% of Propane/Butane (2,5 bar).

### Example 31

### Antiperspirant Spray Emulsion Composition

A sufficient amount of microcapsule slurry E, F, G, H, I, J or K is weighed and mixed in antiperspirant spray emulsion composition (Table 29) to add the equivalent of 0.2% perfume.

TABLE 29
antiperspirant spray emulsion composition

| | Amount |
|---|---|
| Ingredient | (wt %) |
| Polysorbate 65¹⁾ (Part A) | 0.95 |
| Polyglyceryl-2 dipolyhydroxystearate²⁾ | 1.05 |
| (Part A) | |
| Cetyl PEG/PPG-10/1 Dimethicone³⁾ | 2.75 |
| (Part A) | |
| Cyclomethicone⁴⁾ (Part A) | 16.4 |
| Isopropylisostearate⁵⁾ (Part A) | 4.5 |
| Phenoxyethanol⁶⁾ (Part A) | 0.5 |
| Ethylhexylglycerin⁷⁾ (Part A) | 0.2 |
| C12-15 Alkyl Benzoate⁸⁾ (Part A) | 5.65 |
| Silica Silylate⁹⁾ (Part A) | 0.1 |
| Sodium Methylparaben¹⁰⁾ (Part B) | 0.1 |
| Aluminium Chlorohydrate¹¹⁾ (Part B) | 20 |
| Water (Part B) | 44.47 |
| Fragrance (Part C) | 3.33 |

¹⁾Tween 65; trademark and origin: CRODA
²⁾Dehymuls PGPH; trademark and origin: BASF
³⁾Abil EM-90; trademark and origin: BASF
⁴⁾Dow Corning 345 fluid; trademark and origin: Dow Corning
⁵⁾Crodamol ipis; trademark and origin: CRODA
⁶⁾Phenoxyethanol; trademark and origin: LANXESS
⁷⁾Sensiva sc 50; trademark and origin: KRAFT
⁸⁾Tegosoft TN; trademark and origin: Evonik
⁹⁾Aerosil R 812; trademark and origin: Evonik
¹⁰⁾Nipagin mna; trademark and origin: CLARIANT
¹¹⁾Locron L; trademark and origin: CLARIANT

The ingredients of Part A and Part B are weighted separately. Ingredients of Part A are heated up to 60° C. and ingredients of Part B are heated to 55° C. Ingredients of Part B are poured small parts while continuous stirring into A. Mixture were stirred well until the room temperature was reached. Then, ingredients of part C are added. The emulsion is mixed and is introduced into the aerosol cans. The propellant is crimped and added.
Aerosol filling: 30% Emulsion: 70% Propane/Butane 2,5 bar

### Example 32

### Deodorant Spray Composition

A sufficient amount of microcapsule slurry E, F, G, H, I, J or K is weighed and mixed in antiperspirant deodorant spray composition (Table 30) to add the equivalent of 0.2% perfume.

TABLE 30
deodorant spray composition

| | Amount |
|---|---|
| Ingredient | (wt %) |
| Ethanol 95 % | 90.65 |
| Triclosan¹⁾ | 0.26 |
| Isopropyl miristate | 9.09 |

¹⁾Irgasan ® DP 300; trademark and origin: BASF

All the ingredients according to the sequence of the Table 24 are mixed and dissolved. Then the aerosol cans are filled, crimp and the propellant is added (Aerosol filling: 40% active solution 60% Propane/Butane 2.5 bar).

### Example 33

### Antiperspirant Roll-on Emulsion Composition

A sufficient amount of microcapsule slurry E, F, G, H, I, J or K is weighed and mixed in antiperspirant roll-on emulsion composition (Table 31) to add the equivalent of 0.2% perfume.

TABLE 31
antiperspirant roll-on emulsion composition

| | Amount |
|---|---|
| Ingredient | (wt %) |
| Steareth-2¹⁾ (Part A) | 3.25 |
| Steareth-21²⁾ (Part A) | 0.75 |
| PPG-15 Stearyl Ether³⁾ (Part A) | 4 |
| WATER deionised (Part B) | 51 |
| Aluminum Chlorohydrate 50% | 40 |
| aqueous solution⁴⁾ (Part C) | |
| Fragrance (Part D) | 1 |

¹⁾BRIJ 72; origin: ICI
²⁾BRIJ 721; origin: ICI
³⁾ARLAMOL E; origin: UNIQEMA-CRODA
⁴⁾LOCRON L; origin: CLARIAN

Part A and B are heated separately to 75° C.; Part A is added to part B under stirring and the mixture is homogenized for 10 minutes. Then, the mixture is cooled down under stirring; and part C is slowly added when the mixture reached 45° C. and part D when the mixture reached at 35° C. while stirring. Then the mixture is cooled down to RT.

### Example 34

### Antiperspirant Roll-on Composition

A sufficient amount of microcapsule slurry E, F, G, H, I, J or K is weighed and mixed in antiperspirant roll-on composition (Table 32) to add the equivalent of 0.2% perfume.

TABLE 32
antiperspirant roll-on composition

| Ingredient | QUANTITY |
|---|---|
| Water (Part A) | 45 |
| Aluminum Chlorohydrate 50% aqueous | 20 |
| solution¹⁾ (Part B) | |
| Alcohol Denat. (Ethanol 96%) (Part B) | 30 |
| Ceteareth-12²⁾ (Part C) | 2 |
| Ceteareth-30³⁾ (Part C) | 2 |
| Fragrance (Part D) | 1 |

¹⁾LOCRON L; origin: CLARIANT
²⁾EUMULGIN B-1; origin: BASF
³⁾EUMULGIN B-3; origin: BASF

The ingredients of part B are mixed in the vessel then ingredient of part A is added. Then dissolved part C in part A and B. With perfume, 1 part of Cremophor RH40 for 1 part of perfume is added while mixing well

### Example 35

### Antiperspirant Roll-on Composition

A sufficient amount of microcapsule slurry E, F, G, H, I, J or K is weighed and mixed in antiperspirant roll-on emulsion composition (Table 33) to add the equivalent of 0.2% perfume.

TABLE 33
antiperspirant roll-on emulsion composition

| Ingredient | Amount (wt %) |
|---|---|
| Water (Part A) | 50.51 |
| Hydroxyethylcellulose¹⁾ (Part A) | 0.71 |
| Ethanol 95 % (Part B) | 40.40 |
| 1,2-Propylene Glycol (Part B) | 5.05 |
| Triclosan²⁾ (Part B) | 0.30 |
| PEG-40 Hydrogenated castor oil³⁾ (Part C) | 3.03 |

¹⁾Natrosol ® 250 H; trademark and origin: Ashland
²⁾Irgasan ® DP 300; trademark and origin: BASF
³⁾Cremophor ® RH 40; trademark and origin: BASF

Part A is prepared by sprinkling little by little the Hydroxyethylcellulose in the water whilst rapidly stirring with the turbine. Stirring is continued until the Hydroxyethylcellulose is entirely swollen and giving a limpid gel. Then, Part B is poured little by little in Part A whilst continuing stirring until the whole is homogeneous. Part C is added.

### Example 36

### Deodorant Pump without Alcohol Formulation

A sufficient amount of microcapsule slurry E, F, G, H, I, J or K is weighed and mixed in the following composition (Table 34) to add the equivalent of 0.2% perfume.

TABLE 34
deodorant composition

| | Amount |
|---|---|
| Ingredients | (wt %) |
| C12-15 Alkyl | 5 |
| Lactate¹⁾ | |
| Dimethicone²⁾ | 91.6 |
| Cetyl Lactate³⁾ | 1 |
| Octyldodecanol⁴⁾ | 0.8 |
| Triclosan⁵⁾ | 0.1 |
| PERFUME | 1.5 |

¹⁾Ceraphyl 41; trademark and origin ASHLAND
²⁾DOW CORNING 200 FLUID 0.65 cs;
trademark and origin DOW CORNING CORPORATION
³⁾Ceraphyl 28; trademark and origin ASHLAND
⁴⁾Eutanol G; trademark and origin BASF
⁵⁾Irgasan ® DP 300; trademark and origin: BASF

All the ingredients of Table 34 are mixed according to the sequence of the table and the mixture is heated slightly to dissolve the Cetyl Lactate.

### Example 37

### Deodorant Pump with Alcohol Formulation

A sufficient amount of microcapsule slurry E, F, G, H, I, J or K is weighed and mixed in the following composition (Table 35) to add the equivalent of 0.2% perfume.

TABLE 35
deodorant composition

| | Amount |
|---|---|
| Ingredients | (wt %) |
| Ethyl Alcohol (Part A) | 60 |
| PEG-6 Caprylic/Capric | 2 |
| Glycerides¹⁾ (Part A) | |
| Water (Part A) | 35.6 |
| PEG-40 Hydrogenated | 0.4 |
| Castor Oi1²⁾ (Part B) | |
| PERFUME (Part B) | 2 |

¹⁾Softigen 767; trademark and origin CRODA
²⁾Cremophor ® RH 40; trademark and origin: BASF

Ingredients from Part B are mixed together. Ingredients of Part A are dissolved according to the sequence of the Table and are poured into part B.

### Example 38

### Deodorant Stick without Alcohol Formulation

A sufficient amount of microcapsule slurry E, F, G, H, I, J or K is weighed and mixed in the following composition (Table 36) to add the equivalent of 0.2% perfume.

TABLE 36
deodorant composition

| | Amount |
|---|---|
| Ingredient | (wt %) |
| Stearic acid (Part A) | 5.05 |
| 1,2-propylene glycol (Part A) | 41.87 |
| Sodium hydroxide 20% aqueous | 4.24 |
| solution (Part A) | |
| Water (Part A) | 30.30 |
| Tetrasodium EDTA¹⁾ (Part A) | 0.10 |
| Ceteareth-25²⁾ (Part A) | 1.52 |
| PPG-3 Myristyl ether³⁾ (Part A) | 1.52 |
| 1,2-propylene glycol (Part B) | 15.14 |
| Triclosan⁴⁾ (Part B) | 0.25 |

¹⁾Edeta ® B Power; trademark and origin: BASF
²⁾Cremophor ® A25; trademark and origin: BASF
³⁾Tegosoft ® APM; trademark and origin: Evonik
⁴⁾Irgasan ® DP 300; trademark and origin: BASF

All the components of Part A are weighted and heated up to 70-75° C. Ceteareth-25 is added once the other Part A ingredients are mixed and heated. Once the Ceteareth-25 is dissolved, the Stearic Acid is added. Part B is prepared by dissolving the Triclosan in 1,2 Propylene Glycol. Water which has evaporated is added. Slowly under mixing, Part B is poured into part A. To stock, a plastic bag into the bucket is put in to be sealed after cooling. Moulds was filled at about 70° C.

### Example 39

### Anti-Perspirant Stick

A sufficient amount of microcapsule slurry E, F, G, H, I, J or K is weighed and mixed in the following composition (Table 37) to add the equivalent of 0.2% perfume.

TABLE 37
deodorant composition

| | Amount |
|---|---|
| Ingredient | (wt %) |
| Cyclomethicone¹⁾ (Part A) | 55.56 |
| Stearyl A1cohol²⁾ (Part A) | 21.21 |
| PPG-14 Butyl ether³⁾ (Part A) | 2.02 |
| Hydrogenated Castor Oil⁴⁾ (Part A) | 1.01 |
| Aluminium Zirconium | 20.20 |
| tetrachlorohydrex-Gly⁵⁾ (Part B) | |

¹⁾Dow Corning ® 345 Fluid; trademark and origin: Dow Corning
²⁾Lanette ® 18; trademark and origin: BASF
³⁾Tegosoft ® PBE; trademark and origin: Evonik
⁴⁾Cutina ® HR; trademark and origin: BASF
⁵⁾Summit AZP-908; trademark and origin: Reheis

All the components of Part A are weighted, heated up to 70-75° C. and mixed well. Ingredient of Part B is dispersed in Part A. The mixture is mixed and putted into a tick at 65° C.

### Example 40

### Day Cream

A sufficient amount of microcapsule slurry E, F, G, H, I, J or K is weighed and mixed in the following composition (Table 38) to add the equivalent of 0.2% perfume.

TABLE 38
day cream

| Ingredients | % |
|---|---|
| ARLATONE 985 | 5.000 |
| Ethoxylated Fatty | |
| Alcohol Ester | |
| CETYL ALCOHOL | 0.500 |
| TEFOSE 2561 | 4.000 |
| Ceteth-20 (and) Glyceryl | |
| Stearate (and) | |
| PEG-6 Stearate (and) | |
| Steareth-20 | |
| COSBIOL | 1.000 |
| Squalan | |
| MINERAL OIL 30-40 cp | 2.000 |
| Paraffin Oil | |
| PETROLEUM JELLY | 6.000 |
| Petrolatum | |
| WATER deionized | 75.850 |
| PROPYLENE GLYCOL | 5.000 |
| GLYDANT PLUS | 0.150 |
| DMDM Hydantoin (and) | |
| Iodopropynyl | |
| Butylcarbamate | |
| PNC 400 | 0.200 |
| Sodium Carbomer | |
| PERFUME | 0.300 |
| Total | 100.00 |

### Example 41

### Talc Formulation

A sufficient amount of granules 1-5 is weighed and mixed in introduced in a standard talc base: 100% talc, very slight characteristic odor, white powder, origin: LUZENAC to add the equivalent of 0.2% perfume.

### Example 42

### Shower-Gel Composition

A sufficient amount of microcapsule slurry E, F, G, H, I, J or K is weighed and mixed in the following composition (Table 39) to add the equivalent of 0.2% perfume.

TABLE 39
shower gel composition

| | Amount | |
|---|---|---|
| Ingredients | (% wt) | Function |
| WATER deionised | 49.350 | Solvent |
| Tetrasodium EDTA ¹⁾ | 0.050 | Chelating agent |
| Acrylates Copolymer²⁾ | 6.000 | Thickener |
| Sodium C12-C15 Pareth Sulfate ³⁾ | 35.000 | Surfactant |
| Sodium Hydroxide 20% | 1.000 | pH adjuster |
| aqueous solution | | |
| Cocamidopropyl Betaine⁴⁾ | 8.000 | Surfactant |
| Methylchloroisothiazolinone | 0.100 | Preservative |
| and Methylisothiazolinone⁵⁾ | | |
| Citric Acid (40%) | 0.500 | pH adjuster |

10) EDETA B POWDER; trademark and origin: BASF
11) CARBOPOL AQUA SF-1 POLYMER; trademark and origin: NOVEON
12) ZETESOL AO 328 U; trademark and origin: ZSCHIMMER & SCHWARZ
13) TEGO-BETAIN F 50; trademark and origin: GOLDSCHMIDT
14) KATHON CG; trademark and origin: ROHM & HASS

Ingredients are mixed, pH is adjusted to 6-6.3 (Viscosity: 4500cPo+/−1500cPo (Brookfield RV/Spindle #4/20RPM)).

### Example 43

### Shower-Gel Composition

A sufficient amount of microcapsule slurry E, F, G, H, I, J or K is weighed and mixed in the following composition (Table 40) to add the equivalent of 0.2% perfume.

TABLE 40
shower gel composition

| | Amount | |
|---|---|---|
| Ingredients | (% wt) | Function |
| WATER deionized | 52.40 | Solvent |
| Tetrasodium EDTA ¹⁾ | 0.10 | Chelating agent |
| Sodium Benzoate | 0.50 | Preservative |
| Propylene Glycol | 2.00 | Solvent |
| Sodium C12-C15 Pareth Sulfate ²⁾ | 35.00 | Surfactant |
| Cocamidopropyl Betaine³⁾ | 8.00 | Surfactant |
| Polyquaternium-7⁴⁾ | 0.20 | Conditioning agent |
| Citric Acid (40%) | 1.00 | pH adjuster |
| Sodium Chloride | 0.80 | Viscosity adjuster |

¹⁾ EDETA B POWDER; trademark and origin: BASF
²⁾ ZETESOL AO 328 U; trademark and origin: ZSCHIMMER & SCHWARZ
³⁾TEGO-BETAIN F 50; trademark and origin: GOLDSCHMIDT
⁴⁾MERQUAT 550; trademark and origin: LUBRIZOL

Ingredients are mixed, pH is adjusted to 4.5 (Viscosity: 3000cPo+/−1500cPo (Brookfield RV/Spindle #4/20RPM)).

### Example 44

### Shower-Gel Composition

A sufficient amount of microcapsule slurry E, F, G, H, I, J or K is weighed and mixed in the following composition (Table 41) to add the equivalent of 0.2% perfume.

TABLE 41
shower gel composition

| | Amount | |
|---|---|---|
| Ingredients | (% wt) | Function |
| WATER deionized | 50.950 | Solvent |
| Tetrasodium EDTA ¹⁾ | 0.050 | Chelating agent |
| Sodium Benzoate | 0.50 | Preservative |
| Glycerin 86% | 3.50 | Solvent |
| Sodium Laureth Sulfate ²⁾ | 27.0 | Surfactant |
| Polyquaternium-7³⁾ | 1.0 | Conditioning Agent |
| Coco-Betaine⁴⁾ | 6.0 | Surfactant |
| PEG-120 Methyl Glucose trioleate⁵⁾ | 1.0 | Thickener |
| Citric Acid (40%) | 1.0 | pH adjuster |
| Glycol Distearate & Laureth-4 & | 3.0 | Pearlizing agent |
| Cocamidopropyl Betaine⁶⁾ | | |
| Sodium Chloride 20% | 5.0 | Viscosity adjuster |
| PEG-40 Hydrogenated Castor Oil⁷⁾ | 1.0 | Viscosity adjuster |

¹⁾ EDETA B POWDER; trademark and origin: BASF
²⁾ Texapon NSO IS; trademark and origin: COGNIS
³⁾MERQUAT 550; trademark and origin: LUBRIZOL
⁴⁾DEHYTON AB-30; trademark and origin: COGNIS
⁵⁾GLUCAMATE LT; trademark and origin: LUBRIZOL
⁶⁾EUPERLAN PK 3000 AM; trademark and origin: COGNIS
⁷⁾CREMOPHOR RH 40; trademark and origin: BASF

Ingredients are mixed, pH is adjusted to 4.5 (Viscosity: 4000cPo+/−1500cPo (Brookfield RV/Spindle #4/20RPM))

### Example 45

### Hand Dishwash

A sufficient amount of microcapsule slurry E, F, G, H, L, J or K is weighed and mixed in the following composition (Table 42) to add the equivalent of 0.2% perfume.

TABLE 42
Hand dishwash composition

| | Amount | |
|---|---|---|
| Ingredients | (% wt) | Function |
| Linear alkylbenzene | 20 | Anionic surfactant |
| sulfonic acid ⁽¹⁾ | | |
| Diethanolamide ⁽²⁾ | 3.5 | Foam booster |
| Sodium Hydroxide (50%) ⁽³⁾ | 3.4 | pH Adjuster/neutralizer |
| Secondary alcohol | 2.5 | Non-ionic surfactant |
| ethoxolate ⁽⁴⁾ | | |
| Sodium xylene sulfonate | 6.3 | Hydrotrope |
| Water | 64.3 | Solvent |

⁽¹⁾ Biosoft S-118 ®; trademark and origin: Stepan Company
⁽²⁾ Ninol 40-CO ®; trademark and origin: Stepan Company
⁽³⁾ Stepanate SXS ®; trademark and origin: Stepan Company
⁽⁴⁾ Tergitol 15-S-9 ®; trademark and origin: Dow Chemical Company

Water with sodium hydroxide and diethanolamide are mixed. LAS is added. After the LAS is neutralized, the remaining ingredients are added. The pH was Checked (=7-8) and adjusted if necessary.

### Example 46

### Toothpaste Formulation

A sufficient amount of microcapsule slurry R (corresponding to microcapsules H or N except that a flavor is encapsulated instead of a perfume) is weighed and mixed in the following composition (Table 43) to add the equivalent of 0.2% flavor.

TABLE 43
Toothpaste formulation

| | Amount |
|---|---|
| Ingredients | (% wt) |
| Polyethylene glycol 400 | 2.0% |
| Xanthan Gum | 0.60% |
| Sorbitol 70% Solution | 50.0% |
| Sodium Fluoride | 0.220% |
| Sodium Benzoate | 0.20% |
| Water | 15.230% |
| Hydrated Silica¹⁾ | 22.0% |
| Hydrated Silica²⁾ | 7.0% |
| Titanium Dioxide CI77891 | 0.500% |
| Sodium Lauryl Sulfate | 1.250% |
| Flavor | 1.20% |
| TOTAL | 100% |

¹⁾Tixosil 73; trademark and origin:
²⁾Tixosil 43; trademark and origin:

### Example 47

### Dicalcium Phosphate Based Toothpaste Formulation

A sufficient amount of microcapsule slurry R (corresponding to microcapsules H or N except that a flavor is encapsulated instead of a perfume) is weighed and mixed in the following composition (Table 44) to add the equivalent of 0.2% flavor.

TABLE 44
Toothpaste formulation

| | Amount |
|---|---|
| Ingredients | (% wt) |
| Sodium carboxymethyl cellulose | 1.20% |
| Flavor | 1.20% |
| DI/Purified Water | Q.S to Final Wt. |
| Sodium Lauryl Sulfate | 1.30% |
| Glycerine | 20.0% |
| Sodium Saccharin | 0.20% |
| Dicalcium phosphate dihydrate | 36.0% |
| Methylparaben | 0.200% |
| Silica¹⁾ | 3.0% |
| TOTAL | 100% |

¹⁾Aerosil ® 200; trademark and origin:

### Example 48

### Mouthwash Alcohol Free Formulation

A sufficient amount of microcapsule slurry R (corresponding to microcapsules H or N except that a flavor is encapsulated instead of a perfume) is weighed and mixed in the following composition (Table 45) to add the equivalent of 0.2% flavor.

TABLE 45
Mouthwash formulation

| Ingredients | Amount (% wt) |
|---|---|
| Propylene Glycol | 10.0% |
| Flavor | 0.240% |
| DI/Purified Water | Q.S to Final Wt. |
| Poloxamer 407 NF | 0.240% |
| Sodium Lauryl Sulfate | 0.040% |
| Sorbitol 70% Solution | 10.0% |
| Sodium Saccharin | 0.030% |
| Glycerine | 3.0% |
| Sodium Benzoate | 0.10% |
| Sucralose | 0.020% |
| Benzoic Acid | 0.050% |
| TOTAL | 100% |

### Example 49

### Mouthwash Formulation

A sufficient amount of microcapsule slurry R (corresponding to microcapsules H or N except that a flavor is encapsulated instead of a perfume) is weighed and mixed in the following composition (Table 46) to add the equivalent of 0.2% flavor.

TABLE 46
Mouthwash formulation

| | Amount |
|---|---|
| Ingredients | (% wt) |
| Ethyl Alcohol 190 Proof | 15.00% |
| Flavor | 0.24% |
| DI/Purified Water | Q.S to Final Wt. |
| Poloxamer 407 NF | 0.24% |
| Sodium Lauryl Sulfate | 0.04% |
| Sorbitol 70% Solution | 10.00% |
| Sodium Saccharin | 0.03% |
| Glycerine | 3.00% |
| Sodium Benzoate | 0.10% |
| Sucralose | 0.02% |
| Benzoic Acid | 0.05% |
| TOTAL | 100% |

1. Process for preparing a core-shell microcapsule slurry, wherein the process comprises the steps of:
(i) Admixing a salt and optionally a cross-linker into an aqueous solution comprising at least a protein to form an aqueous phase;
(ii) Dispersing an oil phase comprising a hydrophobic material, preferably a perfume oil or a flavor oil, into the aqueous phase to form an oil-in-water emulsion;
(iii) Adding into the oil-in-water emulsion a cross-linker if such a cross-linker has not yet been added in step (i);
(iv) Applying sufficient conditions to induce the cross-linking of the protein so as to form a core-shell microcapsule in the form of a slurry.

2. The process according to claim 1, comprising the steps of:
(i) Admixing a salt into an aqueous solution comprising at least a protein to form an aqueous phase;
(ii) Dispersing an oil phase comprising an hydrophobic material, preferably a perfume oil or a flavor oil, into the aqueous phase to form an oil-in-water emulsion;
(iii) Adding into the oil-in-water emulsion a cross-linker; and
(iv) Applying sufficient conditions to induce the cross-linking of the protein so as to form a biopolymer shell.

3. The process according to claim 1, wherein the protein is used in an amount comprised between 0.5 and 10% based on the total weight of the microcapsules slurry.

4. The process according to claim 1, wherein the protein is chosen in the group consisting of milk proteins, sodium caseinate, calcium caseinate, casein, whey protein, hydrolyzed proteins, gelatins, gluten, pea protein, soy protein, silk protein and mixtures thereof.

5. The process according to claim 4, wherein the protein is a mixture of sodium caseinate and whey protein.

6. The process according to claim 1, wherein the salt added in the aqueous solution of step a) is chosen in the group consisting of CaCl2, NaCl, KCl, LiCl, Ca(NO3)2, MgCl2, and mixtures thereof.

7. The process according to claim 1, wherein the weight ratio between the salt and the protein is comprised between 0.01:1 to 1:1.

8. The process according to claim 1, wherein the cross-linker is an enzyme, preferably transglutaminase.

9. The process according to claim 1, wherein the oil phase further comprises a polyfunctional monomer, preferably a polyisocyanate having at least two polyisocyanate groups.

10. The process according to claim 1, wherein the process comprises after step (iv) further steps consisting of
(v) optionally, adsorption of at least one mineral precursor on the microcapsule shell; and
(vi) applying conditions suitable to induce growth of a mineral layer on the microcapsule shell.

11. The process according to claim 10, wherein the mineral precursor is adsorbed on the microcapsule shell by incubating the core-shell microcapsules in at least one mineral precursor solution, wherein the mineral precursor solution is chosen in the group of iron (II) sulfate solution, iron (III) chloride solution, calcium-based salt solution, phosphate-based salt solution, carbonate based salt solution, titanium-based precursor solution, zinc-based precursor solution, and mixtures thereof.

12. The process according to claim 10, wherein the microcapsules obtained in step (v) are further incubating in a second oppositely charged mineral precursor solution or in a solution to induce mineralization of the mineral precursor of step (v).
